(19) United States
(12) Patent Application Publication
(10) Pub. No.: US 2011/0028730 A1
(43) Pub. Date: Feb. 3, 2011

(54) CYCLIC COMPOUNDS AND THE USE THEREOF AS LIGHT ABSORBERS, LIGHT EMITTERS, OR COMPLEX LIGANDS

(75) Inventors: Martin Könemann, Mannheim (DE); Thomas Geßner, Heidelberg (DE); Rüdiger Sens, Ludwigshafen (DE); Christian Lennartz, Sankt Augustin (DE); Günther Seybold, Neuhofen (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF Aktiengesellschaft, Ludwigshafen (DE)

(21) Appl. No.: 12/897,334

(22) Filed: Oct. 4, 2010

Related U.S. Application Data
(63) Continuation of application No. 10/509,944, filed on Oct. 4, 2004.

(30) Foreign Application Priority Data
Apr. 4, 2002 (DE) 102 14 937.2

(51) Int. Cl. C07F 1/08; C07D 487/22; C07D 498/22

(52) U.S. Cl. 548/101; 548/301.7; 548/218

(57) ABSTRACT

The use of cyclic compounds of the formula (I)

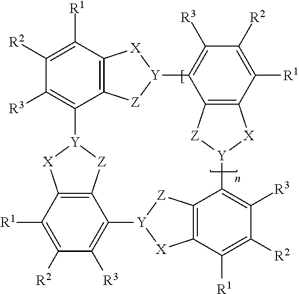

where
n is a number in the range from 1 to 7,
X—Y—Z, in each case independently of one another, is O—C═N, N═C—O, NR⁵—C═N, N═C—NR⁵, N⁺R⁵₂—C═N, N═C—N⁺R⁵₂, O—C═N⁺R⁵, N⁺R⁵═C—O, S—C═N⁺R⁵, N⁺R⁵═C—S, S—C═N, N═C—S,
R¹, R² and R³ each independently are, for example, H or a substituent
   or corresponding heterocyclic compounds in which at least one group —CR¹═, —CR²═, CR³═ is replaced by —N,
R⁵ in each case independently are, for example, H or a substituent
R⁷ in each case independently of one another, are H, C₁₋₁₂-alkyl or C₆₋₁₂-aryl,
or metal complexes of the cyclic compounds or complexes of the cyclic compounds with mineral acids,
chloride, sulfate, bisulfate, phosphate, hydrogen phosphate, nitrate, BF₄⁻ or methanesulfonate being present as opposite ions X⁻ in the case of cationic cyclic structures,
as light absorbers, materials for hole injection layers in OLEDs, light-emitting compounds in OLED, phase-transfer catalysts or synergistic agents for the dispersing of pigments or for optical data storage, is described.

The present invention relates to cyclic compounds, processes for their preparation, their use as photoactive performance chemicals, such as light absorbers or light-emitting compounds, dispersants or as complex ligands, and complexes containing them.

In the context of the present invention, light-absorbing compounds (light absorbers) are usually divided according to the frequency range in which they absorb light. Thus, a distinction is made between UV absorbers, which absorb UV light, V is absorbers (colorants), which absorb visible light, and IR absorbers, which absorb infrared radiation. Furthermore, light absorbers are classified and distinguished on the basis of their solubility or insolubility in the application medium and according to the type of emission of the absorbed energy, for example as heat or as radiation.

Soluble and insoluble compounds which absorb in the UV range and emit the absorbed energy in the form of heat are frequently used as UV absorbers for the purpose of UV protection. Chromophores usually used for these applications are derivatives of triazine, benzophenones, benzotriazoles and cyanoacrylates, as well as ZnO and TiO₂. If the UV radiation is emitted in the form of fluorescent radiation, which as a rule is the case only with compounds soluble in the application medium, optical brighteners which make white materials appear less yellow are obtained. Chromophores used for these applications are in particular benzoxazoles, coumarins and naphthylimides, cf. G. Pritchard, Plastic Additives, Chapmann & Hall, Weinheim 1998.

Compounds which absorb in the visible range of light, are soluble in the application medium and emit their absorbed energy in the form of heat are referred to as dyes. If these compounds soluble in the application medium emit energy in the form of radiation, the term fluorescent dyes is used. Compounds which absorb in the visible range and are insoluble in the application medium are referred to as pigments and emit their energy in the form of heat. Pigments and fluorescent dyes are used for coloring plastics, paper fibers, fibers, etc. Perylene compounds, phthalocyanine compounds, indanthrone compounds, azo compounds, quinophthalone compounds, quinacridone compounds, isoindoline compounds and diketopyrrolopyrrole compounds are usually used for this purpose, cf. W. Herbst, K. Hunger, Industrial Organic Pigments, VCH Weinheim, 1993.

In the case of all photoactive compounds, the potential applications are limited by their long-term stability. Long-term stability is manifested in the stability to light or UV radiation, humidity and heat. This long-term stability is generally accompanied by chemical stability.

In the area of the complex ligands, crown ethers are frequently used as polydentate complexing agents. This is a class consisting of planar macrocyclic polyethers. Frequently, the oxygen atoms are linked by ethylene bridges, in many cases one or more benzene or cyclohexane rings being fused. Some or all of the oxygen atoms of the crown ether may also be replaced by other hetero atoms, such as nitrogen, phosphorus or sulfur. This results in, for example, aza-, phospha- or thia-crown ethers. Polar groups which can occupy the donor position may furthermore be present.

The crown ethers known to date do not have a completely suitable property profile for all complexing tasks. There is therefore still a need for cyclic complex ligands which exhibit novel property profiles.

Synthesis No. 6 (2002), 723 to 725 describes in particular quaterbenzoxazole compounds and quaterbenzimidazole compounds which can be used as complex ligands.

U.S. Pat. No. 5,180,821 describes a cyclic tetrabenzimidazole and a process for its preparation. The preparation is effected by stepwise synthesis of a linear tetramer by means of protective group chemistry. The linear tetramer is then cyclized. Furthermore, a copper complex of the cyclic tetrabenzimidazole product is described. Reference is also made in the document to the prior U.S. Pat. No. 3,481,945, but the process described there led to fluoridine and not, as stated, to cyclic tetrabenzimidazole.

Organic light-emitting compounds are often used in organic light-emitting diodes (OLED). Organic light-emitting diodes (OLED) are generally known and are described, for example, in Angew. Chem. 110 (1998), 416 to 443. The composition of organic light-emitting diodes was described, for example, in C. W. Tang and S. A. Van Slyke, Appl. Phys. Lett. 51 (1987), 913-915, and by M. A. Baldo, M. E. Thompson and S. R. Forrest, Pure Appl. Chem. 71 (1999), 2095-2106. They can be used in many areas, for example in monochrome, multicolor and full color screens, which in turn are used, for example, in automobiles, mobile telephones or notebooks.

The known light-emitting materials for OLEDs, in particular blue and red emitters, still have insufficient long-term stability. The light emission of the emitter materials may be based on fluorescence or phosphorescence. Phosphorescent emitters are described, for example, in WO 01/08230. They are based on heavy metal complexes which have short-lived phosphorescence.

The use of cobalt phthalocyanine-based light absorbers for optical data storage is described in DE-A-101 24 585.

### Synergistic Agents for Pigment Dispersing

EP-A 0554971 describes the use of sulfonated phthalocyanines for preventing the flocculation of finely dispersed phthalocyanine pigments. DE-A 43 25 247 describes carboxylated and sulfonated perylene derivatives in combination with basic polymers for preventing flocculation and for improving rheological properties in highly pigmented finish systems. Sulfonated pigment derivatives are thus used for the surface modification of the pigments and, depending on the formulation, said pigments then have better rheological properties and improved transparency. Further explanations are to be found in DE-A 10303916 and the literature cited there, in particular in the publication Science and Technology of Pigment Dispersions, A four day post graduate intensive course presented by Institute of Materials Science.

In order to avoid changing the color properties of the surface-modified pigments, red sulfonated pigment derivatives are used for the surface modification of perylene pigments (red) or blue sulfonated pigment derivatives for the surface modification of blue pigments, e.g. phthalocyanine (blue) or indanthrone pigments (blue). Various sulfonated pigment derivatives are therefore required for the pigments of different colors.

The object was to find a colorless sulfonated pigment derivative which can be used independently of the color of the pigment to be modified.

This is achieved by the novel sulfonated macrocycles, for example the cycloquaterbenzoxazoles, cycloquaternaphthoxazoles, cycloquaterbenzimidazoles and cycloquaternaphthimidazoles, which exhibit virtually no absorption in the visible range of the spectrum.

It is an object of the present invention to provide cyclic compounds which can be used in particular as photoactive performance chemicals, such as light absorbers or light emitters, or as complex ligands.

We have found that this object is achieved, according to the invention, by the use of cyclic compounds of the formula (I)

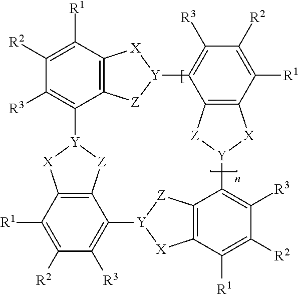

where
n is a number in the range from 1 to 7, preferably from 1 to 3, in particular 1,
X—Y—Z, in each case independently of one another, is O—C═N, N═C—O, NR⁵—C═N, N═C—NR⁵, N⁺R⁵₂—C═N, N═C—N⁺R⁵₂, O—C═N⁺R⁵, N⁺R⁵═C—O, S—C═N⁺R⁵, N⁺R⁵═C—S, S—C═N, N═C—S,
R¹, R² and R³, in each case independently of one another, are H or a substituent from the group consisting of C₁₋₁₂-alkyl, C₁₋₁₂-alkanoyl, C₃₋₇-cycloalkyl, C₆₋₁₂-aryl, C₇₋₁₃-aralkyl, C₇₋₁₃-alkaryl, C₁₋₁₂-alkoxy, C₆₋₁₂-aryloxy, C₁₋₁₇-hydroxyalkyl, a heterocycle, C₆₋₁₂-aroyl, each of which may be substituted, hydroxyl, thiol, halogen, cyano, isocyano, nitro, ammonium, amino, phosphine, phosphine oxide, a sulfonic acid or a derivative thereof, carboxylic acid or a derivative thereof, a derivative of silicon, C₂₋₁₂-alkynyl or C₂₋₁₂-alkenyl, it being possible for the double or triple bonds to be linked directly to the cycloquater skeleton or to be in the chain, a carbamate of the formula —NH—CO—OR⁷, a substituted urea of the formula —NR⁷—CO—NR⁷₂, an alkyl carbonate substituent of the formula —O—CO—OR⁷, a sulfinic acid of the formula —SO—OR⁷ or a derivative thereof, a sulfoxide of the formula —SO—R⁷ or a derivative thereof, phosphonic acid or a salt, ester or amide thereof,
   it also being possible for R¹ and R² and/or R² and R³, in each case independently of one another, to form unsubstituted or substituted fused ring systems comprising from 1 to 3 rings, which may contain hetero atom groups, or to form unsubstituted or substituted alkylene groups which may be interrupted by hetero atom groups, it also being possible for the fused compounds to be substituted as stated above for the radicals R¹, R² and R³,
   it being possible for oxygen atoms in radicals carrying oxygen atoms also to be replaced by sulfur atoms,
   it being possible for on average from 0.05 to 100% of the radicals R¹, R² and R³ present in the molecule to differ from hydrogen,
   or corresponding heterocyclic compounds in which at least one group —CR¹═, —CR²═, —CR³═ is replaced by —N═,
R⁵ in each case independently of one another, are H, unsubstituted or substituted C₁₋₁₂-alkyl, COOH, COO-alkali metal, COO(alkaline earth metal)_{0.5}, COONH₄, SO₃H, SO₃-alkali metal, SO₃(alkaline earth metal)_{0.5}, SO₃NH₄, NR⁷₂, N⁺R⁷₃, 1-pyridino, 4-pyridyl and 4-(1-methyl)pyridinium being suitable substituents, C₆₋₁₂-aryl, C₇₋₁₃-alkylaryl, unsubstituted or substituted C₁₋₁₂-alkanoyl, e.g. formyl, acetyl or chloroacetyl, unsubstituted or substituted C₇₋₁₃-aryloyl, e.g. benzoyl, oligoethylene glycol having 1 to 6 oxygen atoms, oligoethylene glycol ether having 1 to 6 oxygen atoms, imidazoylmethyl or a corresponding radical in which a nitrogen atom is substituted by a C₁₋₁₂-alkyl radical and may carry a positive charge and a C—H group in the ring may be replaced by C—(C₁₋₁₂-alkyl), or (1-C₄₋₆-lactam)methyl, which may be C₁₋₁₂-alkyl-substituted on the ring,
R⁷ in each case independently of one another, are H, C₁₋₁₂-alkyl or C₆₋₁₂-aryl,
and tautomeric structures thereof
or metal complexes of the cyclic compounds or complexes of the cyclic compounds with mineral acids,
chloride, sulfate, bisulfate, phosphate, hydrogen phosphate, nitrate, BF₄⁻ or methanesulfonate being present as opposite ions X⁻ in the case of cationic cyclic structures,
   as light absorbers, materials for hole injection layers in OLEDs, light-emitting compounds in OLED, phase-transfer catalysts and synergistic agents for the dispersing of pigments or for optical data storage.

We have found that the object is preferably achieved by the use of cyclic compounds of the formula (I)

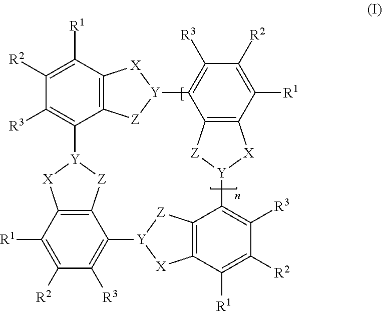

where
n is an integer in the range from 1 to 7,
X—Y—Z, in each case independently of one another, is O—C═N, N═C—O, N═C—NH, S—C═N or N═C—S,
R¹, R² and R³, in each case independently of one another, are H or a substituent from the group consisting of C₁₋₁₂-alkyl, C₁₋₁₂-alkanoyl, C₃₋₇-cycloalkyl, C₆₋₁₂-aryl, C₇₋₁₃-aralkyl, C₇₋₁₃-alkaryl, C₁₋₁₂-alkoxy, C₆₋₁₂-aryloxy, C₁₋₁₂-hydroxyalkyl, a heterocycle, C₆₋₁₂-aroyl, each of which may be substituted, hydroxyl, thiol, halogen, cyano, isocyano, nitro, ammonium, amino, phosphine, phosphine oxide, a sulfonic acid or a derivative thereof, a carboxylic acid or a derivative thereof or a derivative of silicon,
   it also being possible for R¹ and R² and/or R² and R³, in each case independently of one another, to form unsubstituted or substituted fused ring systems comprising from 1 to 3 rings, which may contain hetero atom groups, or to form unsubstituted or substituted alkylene groups which may be interrupted by hetero atom groups,
   it being possible on average for from 0.01 to 12 of the radicals R¹, R² and R³ present in the molecule to differ from hydrogen,
   or corresponding heterocyclic compounds in which at least one group —CR¹═, —CR²═ or —CR³ is replaced by —N═,
   or metal complexes of the cyclic compounds,
   as light absorbers, materials for hole injection layers and emitters in organic light-emitting diodes (OLED) or phase-transfer catalysts
   or as synergistic agents for the dispersing of pigments.
      n may be an average value if a mixture of compounds having different numbers of ring members is present. Otherwise, n is an integer.

We have found that the object is furthermore achieved, according to the invention, by the use of metal complexes of the compounds of the formula (I), as defined above, or as oxidation catalysts.

The compounds of the formula (I) have high stability and long-term stability in said applications. The compounds of the formula (I) can be adapted to the various applications mentioned through the choice of a suitable substitution pattern.

Some of the compounds and complexes used are novel. The present invention therefore also relates to cyclic compounds as defined above, with the exception of compounds where

X—Y—Z in each case is N═C—O, NH—C═N or N═C—NH,
R¹, R² and R³ in each case are H or C₁₋₆-alkyl.

The excepted compounds are described in the synthesis publication mentioned at the outset and, according to an embodiment of the invention, are also not used in the novel applications. This may also apply in particular to the cyclic quaterbenzimidazole.

The present invention also relates to a process for the preparation of these cyclic compounds of the formula (I) by cyclization of compounds of the formula (II)

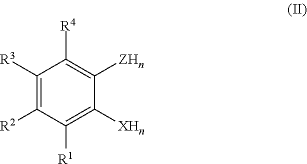

where
R¹, R², R³, X and Z are as defined above for formula (I),
R⁴ is —COOH or a derivative thereof and
n in each case is 1 or 2, to obtain the stoichiometry,
it also being possible for OH groups to be present as alkali metal salt or ammonium salt groups and/or for NH₂ groups to be present in protonated form or in derivative form as —NO, NO₂, —N═N-aryl, ═NOH, ═NH, and it being possible for the cyclization to be carried out in the presence of metal salts, metal powders or Lewis acids as templates.

The cyclization is carried out, for example, in the presence of condensing agents or under dehydrating conditions, it being possible for the cyclization to be carried out in the presence of metal salts or metal powders as templates or in the presence of Lewis acids.

There, R⁴ is a carboxylic acid or a derivative thereof, such as a carboxylic acid salt, an acyl chloride, a carboxamide, a carboxylic ester or a carbonitrile, and

NH₂ is an unsubstituted amine or a derivative thereof, such as an ammonium salt —NH₃⁺, —NO, —NO₂, azo —N═N-aryl, amide —NCO-alkyl or —NHOH (oxime).

The preparation is effected, for example, with heating in an optionally acidic (H₂SO₄, H₃PO₄, polyphosphoric acid) solvent in the presence or absence of metal salt templates, and with or without oxidizing and reducing agents, and, if required, withdrawal of amine, in the presence or absence of an organic solvent.

The process can be carried out in one stage or in two stages, cyclic amides/esters first being prepared and then being further converted into cyclic heteroaromatics, such as oxazoles.

The cyclization is preferably carried out in the presence of condensing agents selected from polyphosphoric acid, (poly)phosphate esters, thionyl chloride and triphenylphosphonium anhydride bis(trifluoromethylsulfonate) or under dehydrating conditions.

According to the invention, the cyclic compounds described above can be used as complex ligands. The present invention also relates to corresponding complexes which contain a complexed metal ion and at least one cyclic compound, as defined above, as complex ligands.

The present invention also relates to processes for the preparation of these complexes of cyclic compounds by the preparation of the cyclic compounds as described in the presence of metal salts or metal powders as templates or by reaction of the cyclic compounds with metal salts or metal powders.

In the above compound of the formula (I), the expression “in each case independently of one another” refers to the individual positions in the molecule in which said groups occur. In each of the positions in the molecule, the groups, independently of one another, may have identical or different meanings. Preferably, each aromatic nucleus in the compounds of the formula (I) has an identical substitution pattern. If X—Y—Z is NH—C═N or N═C—H, it should be noted that these are tautomeric structures. N⁺R⁵₂—C═N may also be, for example, N⁺HR⁵—C═N.

According to an embodiment of the present invention, at least two of the aromatic nuclei are differently substituted.

According to an embodiment of the invention, X—Y—Z and R¹, R² and R³ have the same meanings for all positions.

In heterocyclic systems, one or more groups —CR¹═, —CR²═ and —CR³═ are replaced by —N═. Preferably, not more than two nonneighboring groups are replaced by —N═ in each of the ring-forming aromatic nuclei, independently of one another. Each of the ring-forming aromatic nuclei may also carry —N═ in the ring in an identical manner. Thus, pyridine, pyrimidine or pyridazine structures can be formed. The expression —CR¹═ includes, for example, a carbon atom of the aromatic ring, which, together with the substituent, is replaced by —N═.

R¹, R² and R³ can be chosen so that they do not hinder the cyclization reaction for the preparation of the compounds of the formula (I) or (Ia). Preferred meanings for R¹, R² and R³ are described in more detail below.

X—Y—Z, independently of one another, are preferably selected from O—C═N, N═C—O, NH—C═N, N═C—NH, S—C═N and N═C—S. Preferably, the radicals X—Y—Z, in each case independently of one another, are selected within the three groups O—C═N, N═C—O or NH—C═N, N═C—NH or S—C═N, N═C—S. The middle group comprises tautomeric structures. For the other cases, it is possible to distinguish between six different variants altogether. These are to be explained for O—C═N and N═C—O by way of example, the position of the nitrogen atom on the inside of the cyclic structure or on the outside of the cyclic structure being specified. No nitrogen atoms, 1 nitrogen atom or 2, 3 or 4 nitrogen atoms may be present on the inside. In the case of two internal nitrogen atoms, these can be neighboring or opposite one another, resulting in six structures altogether. The same applies to the systems S—C═N and N═C—S.

R¹, R² and R³, in each case independently of one another, are preferably hydrogen or a substituent from the group consisting of

C₁₋₁₂-alkyl, preferably C₁₋₆-alkyl, in particular C₁₋₃-alkyl, which may be straight-chain or branched or cyclic,
C₆₋₁₂-aryl, preferably phenyl or naphthyl,
C₇₋₁₃-aralkyl, preferably C₇₋₁₁-aralkyl, phenylalkyl radicals in which the alkyl radical may be straight-chain or branched being preferred,
C₇₋₁₃-alkaryl, preferably C₇₋₁₁-alkaryl, alkylphenyl radicals in which the alkyl group may be straight-chain or branched being preferred,
C₁₋₁₂-alkoxy, preferably C₁₋₆-alkoxy, in particular C₁₋₃-alkoxy, it being possible for the alkyl radical to be straight-chain or branched or cyclic,
C₆₋₁₂-aryloxy, preferably phenoxy or naphthyloxy,
C₁₋₁₂-hydroxyalkyl, preferably C₁₋₆-hydroxyalkyl, in particular C₁₋₃-hydroxyalkyl,
it being possible for the above radicals in each case to be further substituted, for example by the following radicals,
hydroxyl,
thiol,
halogen, preferably fluorine, chlorine or bromine, particularly preferably fluorine or
chlorine,
cyano,
isocyano,
nitro,
ammonium,
amino which may be derived from primary, secondary or tertiary amino groups which have alkyl or aryl radicals which correspond to the above definition of alkyl radicals and aryl
radicals,
phosphine or phosphine oxide, it being possible for these radicals to contain alkyl substituents or aryl substituents as described above,
a sulfonic acid or a derivative thereof, it being possible for the derivatives to be acid halides, acid amides or acid esters,
a carboxylic acid or a derivative thereof, it being possible for the derivatives to be acid halides, acid amides or acid esters,
a derivative of silicon, e.g. silyl, it being possible for different silyl groups which may have hydrogen atoms, alkyl radicals and/or alkoxy radicals to be present.

Said alkyl radicals may be interrupted by from 1 to 10 nonneighboring oxygen atoms, resulting in ether structures. R¹ and R² and/or R² and R³, in each case independently of one another, may also form unsubstituted or substituted fused ring systems comprising from 1 to 3 rings, which may contain hetero atom groups. Preferably, each such fused ring system contains 1 or 2 further ring systems in addition to the aromatic nucleus shown in the formula (I). Different fused systems may be present on the four aromatic nuclei of the formula (I). The fused ring system may be substituted as described above, it being possible for all suitable substituents from alkyl substituents to silyl groups to be present. The fused ring systems may furthermore contain hetero atom groups, so that heteraryl groups are formed. Examples of suitable parent structures and fused ring systems are shown below.

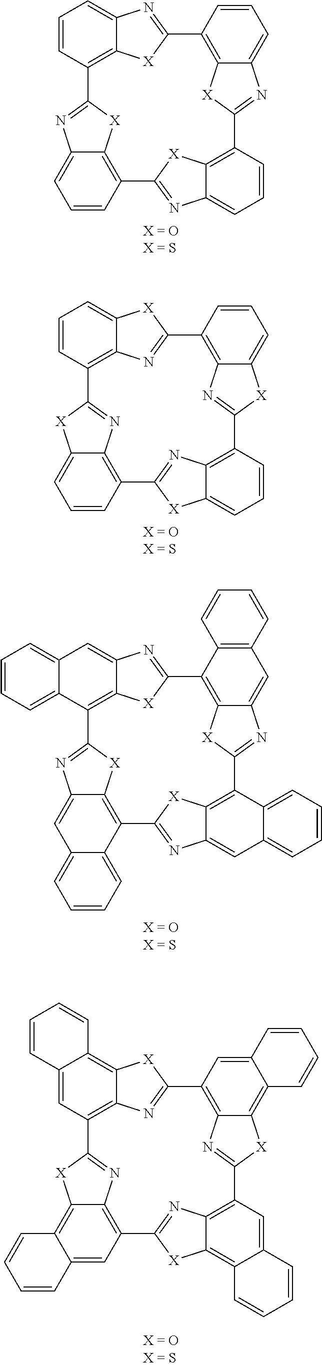

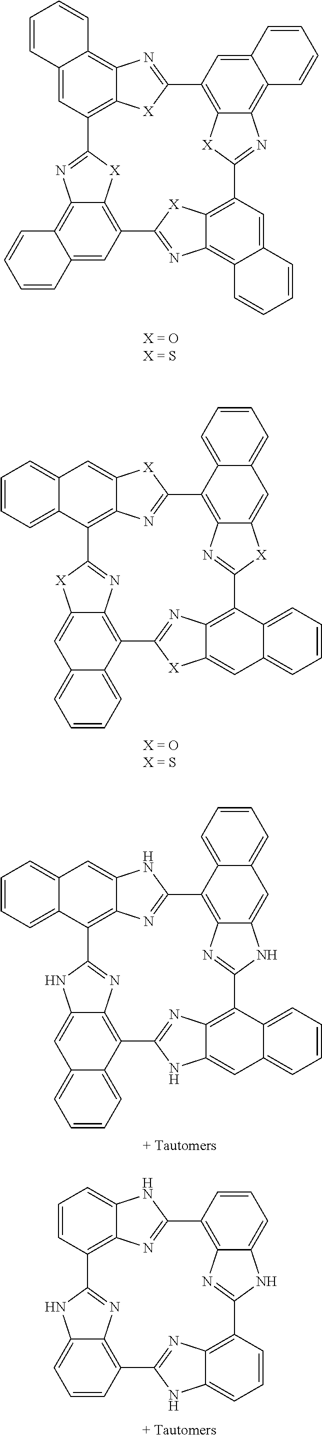

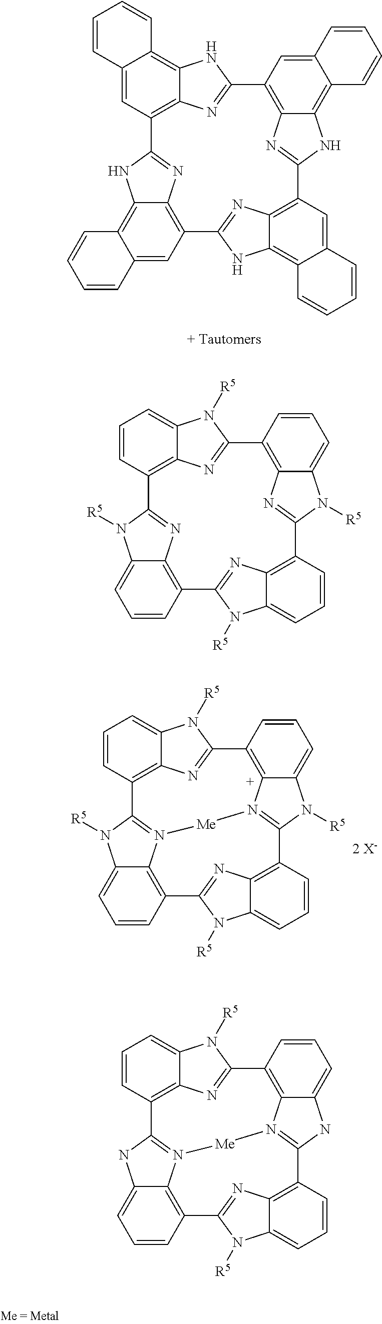

The fused ring systems can be linked to one another in a suitable manner for each of the four aromatic nuclei of the formula (I). For example, linear or nonlinear fused structures can be formed.

R¹ and R² and/or R² and R³, in each case independently of one another, may also form unsubstituted or substituted alkylene groups which may be interrupted by hetero atom groups. This gives aliphatic or heteroaromatic rings or ring systems. The distance between linkage points of the alkylene groups is therefore preferably chosen so that a 5-, 6- or 7-membered ring structure results. The alkylene groups are preferably of 2 to 10, particularly preferably 3 to 10, carbon atoms, a 5-, 6- or 7-membered ring preferably being formed. The alkylene groups can be straight-chain or branched. Suitable hetero atoms are oxygen, sulfur and nitrogen atoms (NH).

In a molecule of the formula (I), R¹, R² and R³ may also form different cyclic structures for different molecular groups. For example, fused structures may be present alongside aliphatic ring structures.

According to the invention, all of the radicals R¹, R² and R³ may be hydrogen atoms. In this case, unsubstituted ring systems are present. If substituents are present, on average from 0.01 to 12 of the radicals R¹, R² and R³ present in the molecule may differ from hydrogen. On average, preferably from 1 to 8 substituents are present. In the case of the low degrees of substitution, the compounds are partly substituted. This means that substoichiometric amounts of substituents are present, so that only some of the molecules are substituted. In this case, mixtures of unsubstituted and substituted compounds of formula (I) are present.

According to an embodiment of the invention, any of the abovementioned radicals can be substituted by the others of the abovementioned radicals. Fused ring systems, too, can be further substituted. A fused ring system may be substituted, for example, by alkyl radicals, aryl radicals, halogen atoms, amino groups, etc.

In the compounds of the formula (I), carbocyclic aromatic or carbocyclic nonaromatic groups, aromatic heterocycles or nonaromatic heterocycles or mixtures thereof may therefore be present. Fused rings may be linear or branched.

The present invention also relates to compounds where n is greater than 1, since these compounds form as byproducts in the cyclization in PPA. Angewandte Chemie (Issue 114/8 1480-1483, 2002) reports on octacyclic pyrroles having IR-absorbing properties which are prepared by cyclization in H₂SO₄. There, the mineral acid acts as a template and promotes the formation of the cyclo[8]pyrroles. Similar cyclization products can also form in the chemical according to the invention, which products can also be used as photoactive performance chemicals, especially as compounds which are transparent in the visible range. Preliminary calculations show deviations from planarity in the case of n=2,3, etc.

A complexed metal ion and at least one cyclic compound of the formula (I) as a complex ligand are present in the novel complexes. The complex metal ions may be derived from metals of the main groups, from the transition metals and from the rare earths of the Periodic Table of the Elements. The following elements may be mentioned in particular:

Li, Na, K, Rb, Cs, Fr, Be, Mg, Ca, Sr, Ba, Ra, Sc, Y, La, Ac, Ce, Pr, Nd, Pm, Sm, Eu, Gd, Tb, Dy, Ho, Er, Tm, Yb, Lu, Th, Pa, U, Np, Pu, Am, Cm, Bk, Cf, Es, Fm, Md, No, Lr, Ti, Zr, Hf, V, Nb, Ta, Cr, Mo, W, Mn, Tc, Re, Fe, Ru, Os, Co, Rh, Ir, Ni, Pd, Pt, Cu, Ag, Au, Zn, Cd, Hg, B, Al, Ga, In, Tl, Si, Ge, Sn, Pb, As, Sb, Bi, Se, Te and Po.

Preferred metals for OLED applications are Eu, Tb, Re, Ru, Os, Ir, Pt, Cu, Au, Tl, Pb and Bi.

Preferred metals for oxidation catalysts are Co, Fe, Mn, Cr and R^{u}.

Particularly preferably, the novel complexes contain one of the cyclic compounds of the formula (I).

The preparation of the novel cyclic compounds of the formula (I) or of the corresponding cyclic compounds used according to the invention is effected by cyclization of compounds of the formula (II)

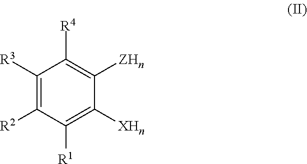

where
R³, R², R³, X and Z are as stated above,
R⁴ is —COOH or a derivative thereof and
n in each case is 1 or 2, in order to obtain the stoichiometry,
it also being possible for O-alkali metal groups to be present instead of OH groups. The cyclization can preferably be carried out in the presence of condensing agents selected from polyphosphoric acid, (poly)phosphate esters, thionyl chloride and triphenylphosphonium anhydride bis(trifluoromethanesulfonate) or under dehydrating conditions.

In principle, the preparation of said tetracyclic parent structures is effected from four identical or different aromatic precursors which may carry the substituents R¹, R² and R³.

Preferably, derivatives of phenolic alcohols (e.g. O-silyl) and aromatic amines (e.g. N-carbamate) are ortho and meta to a carboxylic acid, a carboxylic ester or a carboxamide, it being possible for both the derivative of the phenolic alcohol, which is cleavable under acidic conditions, and the derivative of the aromatic amine to be ortho to the carboxylic acid derivative.

The synthesis is carried out in general under dehydrating conditions, for example in the presence of polyphosphoric acid or concentrated sulfuric acid, with removal of water or alcohol by distillation or with the use of thionyl chloride.

In the above compound of the formula (II), R⁴ is a carboxylic acid radical or a derivative thereof. The derivative is preferably an acid chloride, an ester, an amide, a salt or another corresponding carboxylic acid derivative. Esters are preferably esters of lower alkanols, such as C₁₋₄-alkanols. Amides are preferably derived from ammonia or primary alkylamines.

If the cyclization is carried out with heating in polyphosphoric acid, the procedure is as a rule carried out under an inert gas atmosphere (nitrogen).

The cyclization can be carried out in the absence of a solvent or in a solvent. Suitable solvents are, for example, sulfolane, methylene chloride and ethylene chloride. These may also be mixed with polyphosphoric acid. The reaction can be carried out at room temperature or elevated or lowered temperatures. The reaction temperature is chosen as a function of the reactivity of the components used in each case.

It is also possible to add Lewis acids, e.g. BF₃ Et₂O, etc. The reaction may also be carried out in the presence of metals or metal salts which can act as a template or catalyst or as a reducing agent. For example, the cyclization can be carried out in the presence of zinc chloride or copper sulfate. Metal-containing cyclic structures may also be formed.

According to the invention, the cyclic compounds of the formula (I) and their metal complexes are used as light absorbers or as light-emitting compounds in organic light-emitting diodes (OLED) or as oxidation catalysts (only metal complexes). As light absorbers, they are preferably UV absorbers, V is absorbers and/or IR absorbers. They may be used in soluble, partly soluble or insoluble form in an application medium. The use as an absorber depends on the position of the absorption band of the respective compound. The compounds of the formula (I) form a photoactive system which absorbs light of the corresponding wavelength and emits it in the form of heat or fluorescent radiation. If the compounds are used for pigment applications, they preferably have a mean particle size of from 5 to 1000 nm. Mixtures of the compounds in the form of solid solutions and also mixtures of the compounds with other colorants/pigments chromophores, such as phthalocyanines, may be used.

As UV absorbers, they are preferably used as UV screening agents which convert UV light into heat or as optical brighteners which convert UV light into visible light. They are used as UV absorbers, for example, in cosmetics (sunscreens, for example also in clothing), in automotive top coats, in wood preservation coats, in films, in plastics (for example polystyrene, polycarbonate, polyolefins and also in ABS and ASA plastics or PET). In the plastics applications, either the plastic or the contents of plastics containers can be protected. This applies in particular to transparent or translucent plastics bottles. The same also applies to fibers which are produced from the plastics and are used, for example, for the production of clothing (for example polyamides). Optical brighteners are used, for example, in detergents, textiles or plastics. The plastics can, for example, also be further processed to give films.

If the cyclic compounds are V is absorbers, they may be present as soluble dyes or as insoluble pigments. The soluble dyes can convert visible radiation either into heat or, in the form of fluorescent dyes, into light. As pigments, they convert the light in particular into heat. As colored pigments, they are used for coloring polymeric materials, such as surface coatings and finishes, plastics and printing inks in a large number of applications, for example in the automotive sector.

If the compounds are soluble in organic solvents, polymers or water, they can be used as soluble UV absorbers or V is absorbers (dyes) in the above applications.

The compounds soluble in water, organic solvents or polymeric materials, in particular sulfonic acids, sulfonic esters, sulfonamides, the alkyl- and aryl-substituted members and metal complexes of said compounds can, depending on the position of the absorption maximum, be used as UV-absorbing substances which emit the absorbed energy either in the form of heat (soluble UV absorbers) or in the form of light (optical brighteners). They can be used as dyes for coloring textiles, plastics or paper fibers and as pigment dispersing additives.

Soluble UV absorbers can be used, for example, in cosmetic formulations, in automotive top coats, in wood preserving coats or in films.

Optical brighteners can be used for brightening paper, natural textile fibers or plastics fibers, in detergents, for the optical brightening of plastics, etc.

The novel cyclic compounds or the metal complexes thereof, in particular sulfonic acids, sulfonic esters, sulfonamides, etc., can be used as dispersing additives (synergistic agents) for all known (organic) pigments. As colorless synergistic agents, they can be combined with a large number of different pigments without the color being influenced by their natural color. They can be used, for example, in solvent-containing high-solids finishes.

Nanoparticulate ZnO (De19907704, EP0449 888) is available as a pigmentary inorganic UV absorber for cosmetics, finish and plastic. However, these have the disadvantage that, in the event of insufficient fineness, they scatter white light, giving rise to a milky appearance. Insufficient fineness occurs in the case of an excessively large particle size, either the primary particles being too large or the dispersed state being insufficient. The claimed compounds have an absorption spectrum similar to that of ZnO, but the organic pigments scatter to a lesser extent in the formulation described.

The present invention also relates to the use of the light absorbers for coloring high molecular weight organic materials, for example polymers and similar materials. The present invention also relates to thermoplastic molding materials, finishes and coating compositions which contain the light absorbers in conventional amounts.

According to a further embodiment of the invention, the novel compounds can be used in OLEDs. They may be suitable in particular as emitters in the emitter layer or for the hole injection layer between anode and hole conductor layer and, for example, can replace copper phthalocyanine in the last-mentioned application. An advantage over copper phthalocyanine is a lower absorption in the visible spectral range. Furthermore, heavy metal-containing complexes can be used as triplet emitters in OLEDs. Metal-free and metal complexes of the novel compounds are used for OLED applications, preferably used metals being Cu, Zn and/or Pt.

The novel compounds can furthermore be used in the form of metal complexes as oxidation catalysts. Particularly preferred oxidation catalysts are complexes of the novel compounds with metals selected from Co, Fe, Mn, Cr, Ru and mixtures thereof.

The preparation of the novel compounds is explained in more detail below with reference to synthesis schemes and examples.

Explanations of the synthesis of the asymmetric cycloquater compounds via peptide linkage reactions:

The linear peptides or esters are prepared from the individual building blocks of the formula (II) by standard reactions using standard protective groups. Relevant examples can also be found in Amino Acid and Peptide Synthesis, John Jones, Oxford University Press 1992.

The cyclizations are effected by the method of U. Schmidt at phase boundaries in order to avoid high dilutions.

Relevant literature can be found in:
U. Schmidt et al. Synthesis 1992, 1248-1254
U. Schmidt et al. Synthesis 1992, 1025-1030
U. Schmidt et al. Tetrahedron Lett. 29 (1988), 4407-4408
U. Schmidt et al. Synthesis 1987, 236-241
U. Schmidt et al. J. Org. Chem. 47 (1982), 3261-3264
U. Schmidt et al. Angew. Chem. Int. Ed. Engl. 20 (1981), 280-281
   CBz is benzyloxycarbonyl
   Boc is tert-butoxycarbonyl
   TBS is tert-butyldimethylsilyl

For further protective groups and their attachment and removal, cf.: T. W. Greene and P. G. M. Wuts, Protective Groups in Organic Synthesis, 1991, John Wiley & Sons.

For further abbreviations, see the same reference.

The first two schemes relate to ring structures which have no internal oxygen atoms in the five-membered rings. They are followed by schemes 1, 2, 3 and 4 internal oxygen atoms. Radicals 1, radicals 2, radicals 3 and radicals 4 describe possible substituents of the ring systems.

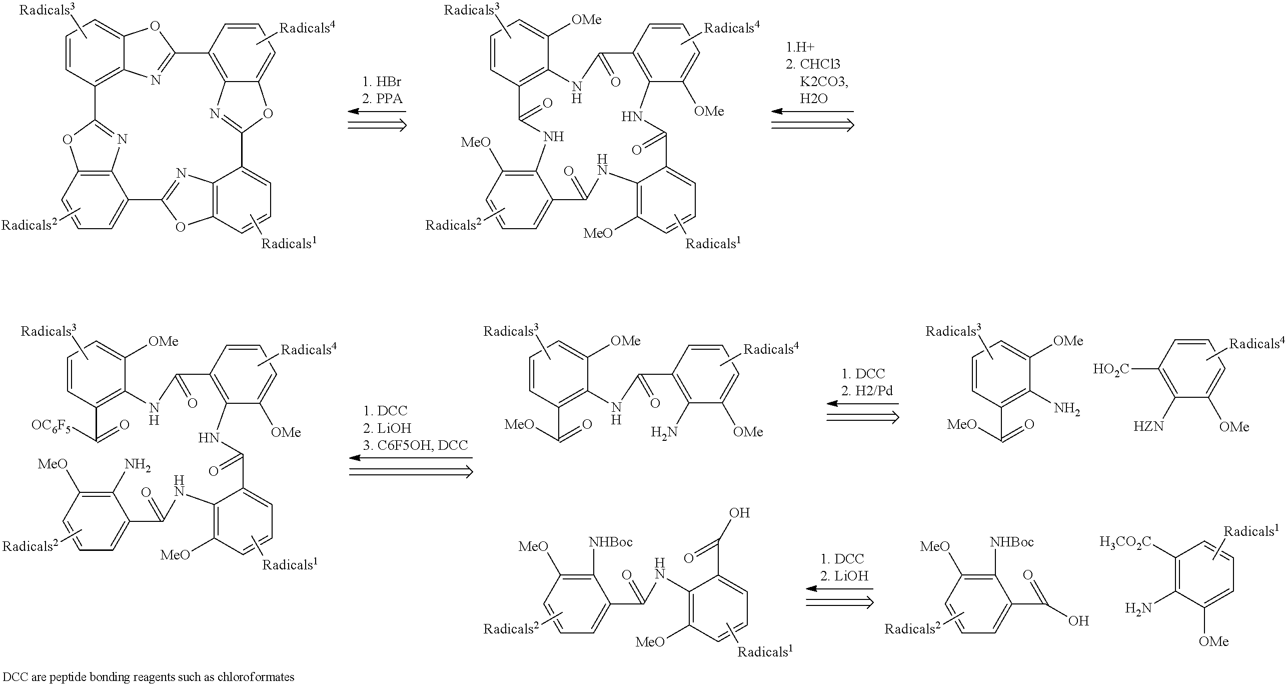

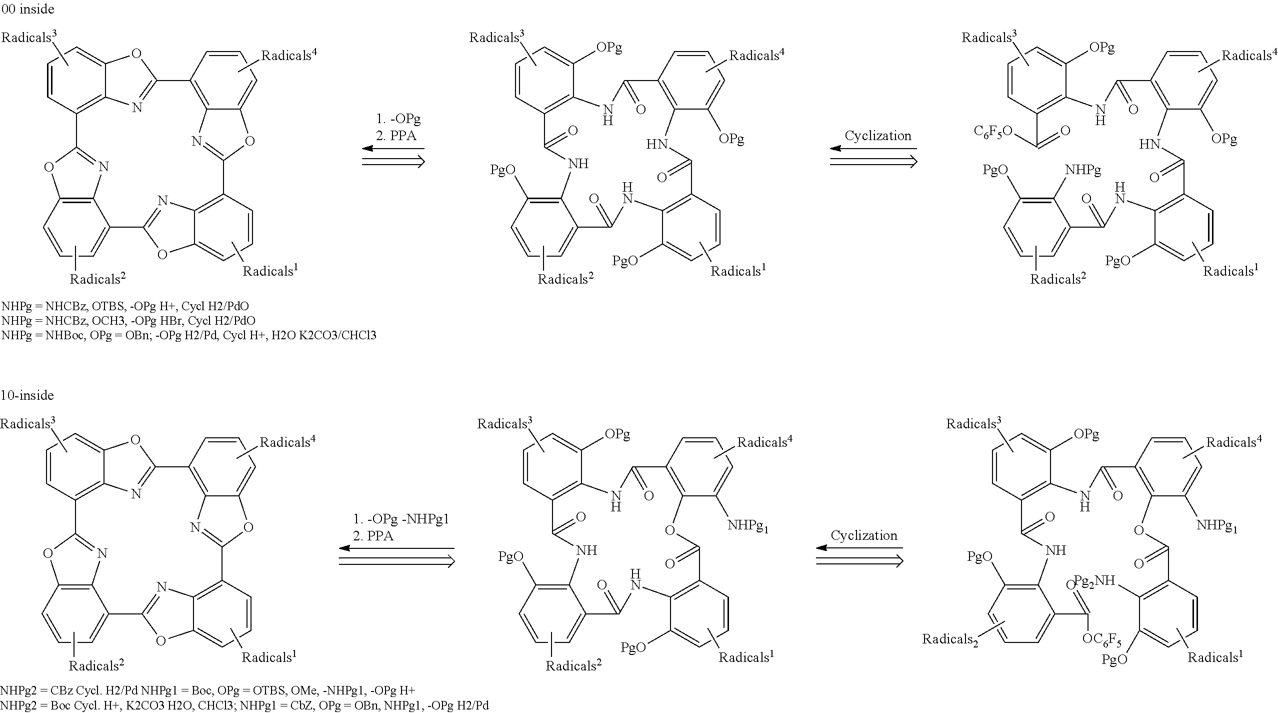

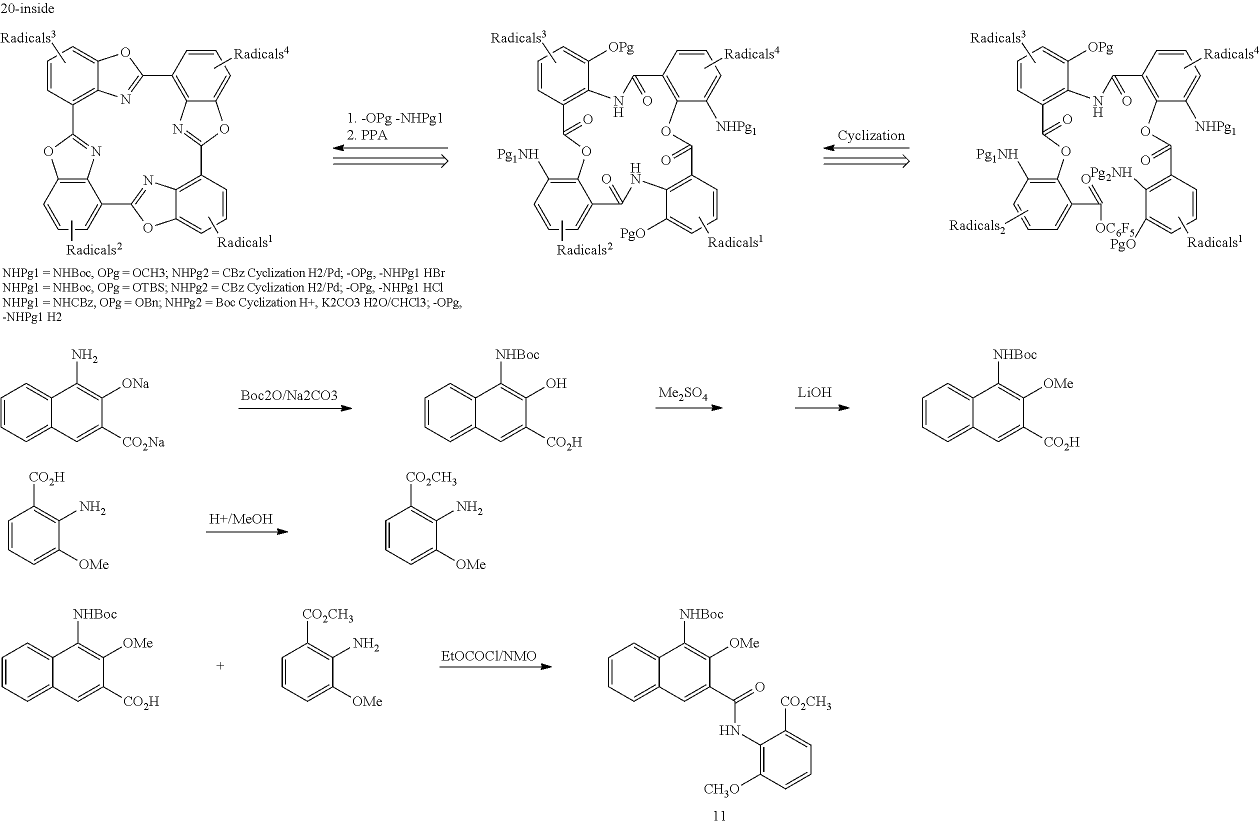

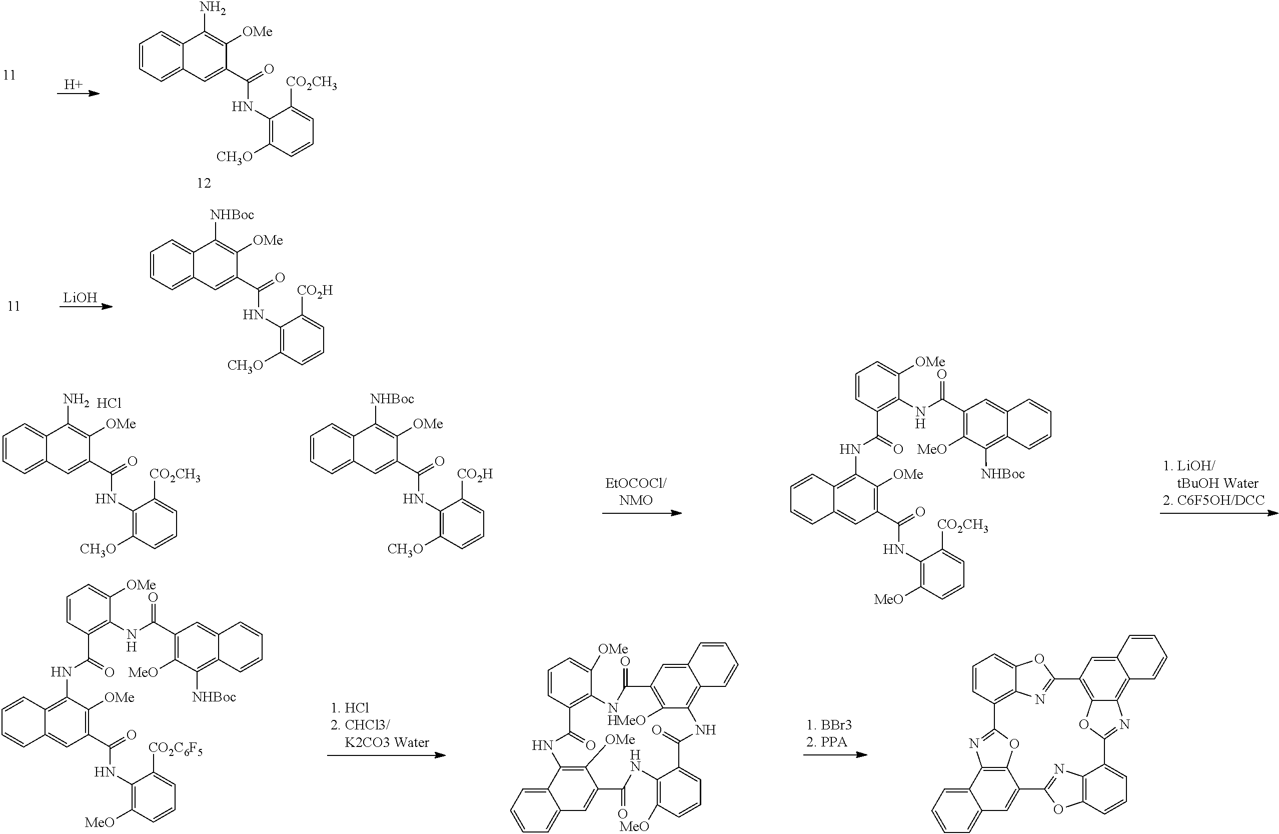

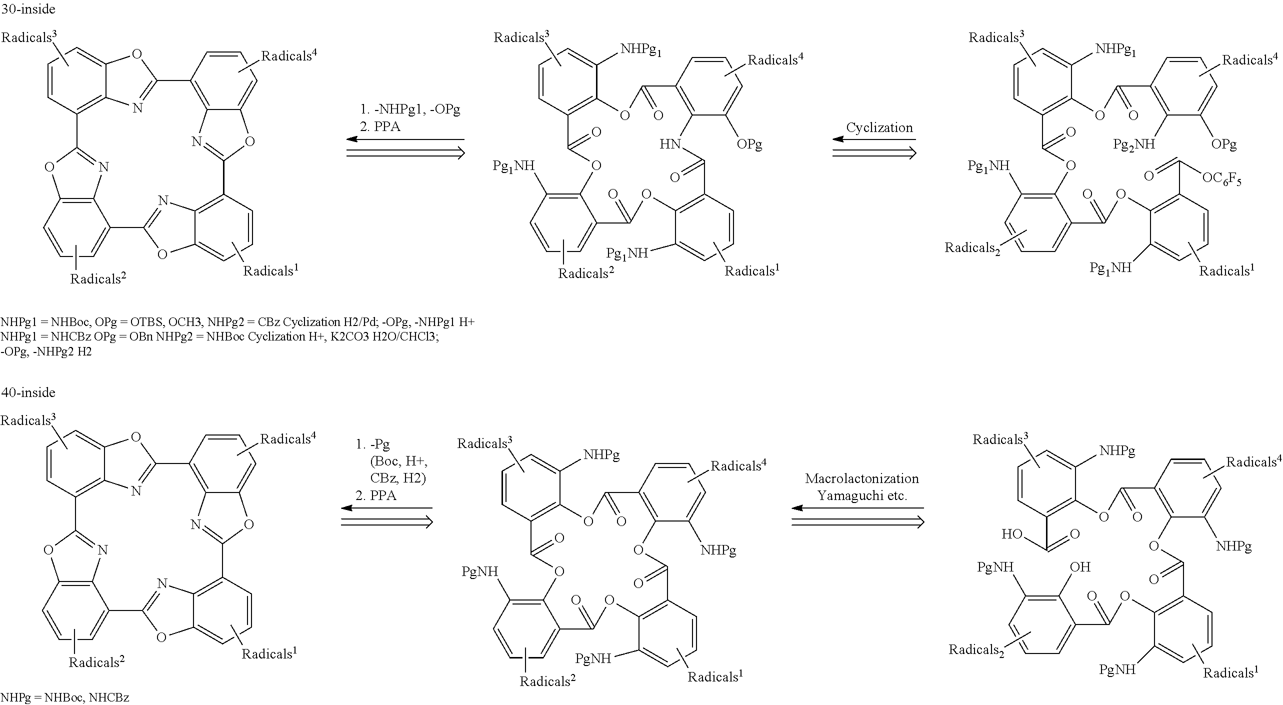

The examples which follow illustrate the invention:

### EXAMPLES

### Example 1

### Cyclo-2,4′:2′,7″:2″,4′″:2′″,7-quaterbenzimidazole/

### Cyclo-2,4′:2′,7″(4″):2″,4′″(7′″):2′″,7″″(4″″):2″″,4(7)-quinquebenzimidazole

20.7 g (122 mmol) of ammonium 2,3-diaminobenzoate were introduced in portions into 240 g of 85% strength polyphosphoric acid at 100° C. with stirring and under nitrogen. Thereafter, the reaction solution was heated to 160° C. with evolution of gas (ammonia) and kept at this temperature for 24 hours. After the solution had been cooled to 60° C., it was poured into 400 ml of ice water, a precipitate being obtained. The precipitate was filtered off with suction and washed with 200 ml of water. (The acidic mother liquor was worked up to obtain the cycloquinquebenzimidazole; see further below.) The moist filter residue was suspended in 500 ml of water. The suspension was made alkaline with ammonia solution and then filtered. The solid was washed with water and dried under reduced pressure at 70° C. 14.2 g of black-brown crude product were obtained. For purification, 14.0 g of crude product were introduced into 280 g of 96% strength sulfuric acid at 10-20° C. in the course of 2 hours. A yellowish solid was precipitated by dropwise addition of 250 ml of water in the course of 2 hours. After stirring overnight at room temperature, the suspension was filtered over a glass frit. The solid was washed with 200 g of 50% strength sulfuric acid. The filter residue was suspended in 400 ml of ice water, filtered off with suction, washed with water and dried at 60° C. under reduced pressure (4.6 g). In a second purification step, this product purified once by precipitation was dissolved in 90 g of 96% strength sulfuric acid at 10-20° C. After stirring for two hours, 200 g of 50% strength sulfuric acid were added dropwise in the course of 2 hours. The suspension was stirred overnight, then filtered off with suction over a glass frit and washed with 50 g of 60% strength sulfuric acid. The filter residue was suspended in 400 ml of water, and sodium hydroxide solution was added to pH 12. The solid was filtered off with suction, washed with water and dried at 85° C. under reduced pressure from an oil pump.

Yield: 1.32 g (9.3%) of greenish yellow microcrystals

According to elemental analysis, the macrocycle crystallized with half a mole equivalent of water

| C₂₈H₁₆N₈•0.5 H₂O | Calc. | C 71.03 | H 3.62 | N 23.67 |
|---|---|---|---|---|
| M = 464.49 + 0.5 × 18.02 = | Found | C 70.8 | H 3.47 | N 23.5 |
| 473.50 | | | | |

MALDI-MS: [M+H]⁺=465.2

UV/Vis: λₘₐₓ (1 g ε)=356 (S), 320 (4.82), 264 (4.58), 220 nm (4.33) in concentrated sulfuric acid, λ_{fluor.}=464 nm in formic acid

The acidic mother liquor was brought to pH 8-9 with sodium hydroxide solution, the resulting precipitate being filtered off with suction, washed with water and dried at 70° C. under reduced pressure. 1.8 g of brownish solid were obtained, and said solid was recrystallized twice from ethylene glycol.

Yield: 1.48 g (8%) of colorless microcrystals

According to elemental analysis and ¹H-NMR in D₆-DMSO, the macrocycle crystallized with 1.5 mole equivalents of water.

| C₃₅H₂₀N₁₀•1.5 H₂O | Calc. | C 69.18 | H 3.82 | N 23.05 |
|---|---|---|---|---|
| M = 580.61 + 1.5 × 18.02 = | Found | C 69.3 | H 3.47 | N 23.1 |
| 607.63 | | | | |

MALDI-MS: [M+H]⁺=581.2

IR (KBr): 3398(s), 3185 (2), 1621, 1537, 1484, 1425 (s), 1368, 1319, 1280, 1238 (s), 1063, 930, 860, 799 (s), 750 (s) cm⁻¹

¹H-NMR (400 MHz, D₆-DMSO): 13.1 (mc; 4H, NH), 12.05 (mc; 1H, NH) 7.8 (mc: 15H, aromatic H), 3.4 (s; 3H, H₂O)

UV/Vis: λₘₐₓ (1 g ε)=354 (S), 310 (4.82), 256 (4.57), 220 nm (4.43) in concentrated sulfuric acid, λₘₐₓ (1g c)=354 (S), 314 nm (4.84) in ethanol,

λ_{fluor.}=461 nm in formic acid

### Example 2

### Bis(2-amino-3-carboxyphenylammonium) hydrogen phosphate (a)

47.50 g (281 mmol) of ammonium 2,3-diaminobenzoate in a solution of 675 ml of acetone and 225 ml of water were refluxed. 32.37 g (281 mmol) of 85% strength o-phosphoric acid were added dropwise at the boil while stirring in the course of 30 minutes. The solution was stirred for a further 20 minutes and filtered while hot. The filtrate was evaporated to dryness. Drying under reduced pressure at 60° C. gave 73.2 g of reddish powder (m.p. 204/205° C. with decomposition), which was recrystallized from 1250 ml of water.

Yield: 39.3 2 (69.5%) of dark red crystals; m.p. 207° C. (decomposition)

| | | | | | |
|---|---|---|---|---|---|
| C₁₄H₁₉N₄O₈P | Calc. | C 41.80 | H 4.76 | N 13.93 | P 7.70 |
| M = 402.30 | Found | C 41.9 | H 4.7 | N 14.0 | P 7.4 |

### Cyclo-2,4′:2′,7″:2″,4′∴:2′″,7-quaterbenzimidazole/

### Cyclo-2,4′:2′,7″(4″):2″,4′(7′∝):2′″,7″″(4″″):2″″, 4(7)-quinquebenzimidazole (b)

20.92 g (52.0 mmol) of bis(2-amino-3-carboxyphenylammonium) hydrogen phosphate were introduced in portions into 240g of 85% strength polyphosphoric acid at 100° C. with stirring and under nitrogen. Thereafter, the reaction solution was heated to 150° C. and kept at this temperature for 24 hours. After the solution had been cooled to 60° C., it was poured into 1000 ml of ice water, a precipitate being obtained. The precipitate was filtered off with suction and washed with 500 ml of water. (The acidic mother liquor was worked up to give cycloquinquebenzimidazole, see further below.) The crude product was purified as described under 1) by precipitation in sulfuric acid.

Yield: 1.23 g (10%) of greenish yellow microcrystals

The work-up of the acidic mother liquor was effected as described under 1).

Yield: 0.54 g (3.4%) of colorless microcrystals

### Example 3

### 4,12-Dimethylcyclo-2,4′:2′,7″:2″,4′″:2′″,7-quaterbenzimidazole

0.340g (2.40 mmol) of methyl iodide was added dropwise to a suspension of 0.465 g (1.00 mmol) of cycloquaterbenzimidazole and 0.652 g (2.00 mmol) of cesium carbonate in 80 ml of anhydrous dimethylformamide with stirring at room temperature. The suspension was stirred for 19 hours at room temperature. The solid was filtered off with suction, washed with dimethylformamide and water and dried at 60° C. under reduced pressure (0.33 g). The solid was recrystallized from 120 ml of N-methyl-2-pyrrolidone, filtered off with suction, washed with N-methyl-2-pyrrolidone, isopropanol and tert-butyl methyl ether and sucked dry. Solvent residues were removed under reduced pressure from an oil pump at 200° C.

Yield: 0.205 g (42%) of yellowish microcrystals

| | | | | |
|---|---|---|---|---|
| C₃₀H₂₀N₈ | Calc. | C 73.16 | H 4.09 | N 22.75 |
| M = 492.54 | Found | C 72.9 | H 4.3 | N 22.7 |

MALDI-MS[M+H]⁺: 493.2

¹H-NMR (500 MHz; D₂SO₄): 7.93 (mc; 8H, aromatic-H), 7.70 (t: 4H, aromatic-H), 3.97 (s; 6H, N—CH₃)

¹³C-NMR (500 MHz; D₂SO₄): 148.51 (s), 148.21 (s), 136.66 (s), 134.27 (s), 131.98 (d), 131.68 (d), 131.48 (s), 131.26 (d), 131.07 (d), 130.31 (s), 123.39 (d), 121.98 (d), 109.85 (s), 109.33 (s), 34.91 (q)

UV/Vis: λₘₐₓ (1 g ε)=350 (S), 318 (4.84), 262 nm (4.59) in concentrated sulfuric acid

λₘₐₓ (1 g ε)=390 (S), 340 nm in dimethylformamide

IR (KBr): 3384, 1463, 1435, 1415, 1326, 1290, 1281, 1260, 795, 752, 739, 717, 661 cm⁻¹

### Example 4

### 4,8,12,16-Tetramethylcyclo-2,4′:2′,4″:2′,4′″:2′″,4-quaterbenzimidazole

0.465 g (1.00 mmol) of cycloquaterbenzimidazole was dissolved in 50 ml of dimethylformamide at room temperature with addition of 0.448 g (4.00 mmol) of potassium tert-butylate. After addition of 0.848 g (6.00 mmol) of methyl iodide, the solution was stirred for 24 hours at room temperature. The solid was filtered off with suction, washed with dimethylformamide and water and dried at 60° C. under reduced pressure.

### Example 5

### 4,8,12,16-Tetramethylcyclo-2,4″:2′,4″:2″,4′″:2′″,4-quaterbenzimidazole

0.914 g (6.00 mmol) of 1,8-diazabicyclo[5.4.0]undec-7-ene was added dropwise to a suspension of 0.465 g (1.00 mmol) of cycloquaterbenzimidazole in 50 ml of dimethyl carbonate at room temperature. After addition of 1.477 g (4.00 mmol) of tetra-n-butylammonium iodide, the reaction mixture was refluxed for 91 hours with stirring. After cooling, the solid was filtered off with suction, washed with dimethyl carbonate, acetone and water and dried at 60° C. under reduced pressure (0.30 g).

### Example 6

### Copper cyclo-2,4″:2′,7″:2″,4′″:2′″,7-quaterbenzimidazole

4.4 g (26 mmol) of ammonium 2,3-diaminobenzoate and 1.03 g (6.5 mmol) of anhydrous copper sulfate were introduced into 120g of 85% strength polyphosphoric acid at 100° C. with stirring and under nitrogen. Thereafter, the reaction solution was heated to 160° C. with evolution of ammonia and kept at this temperature for 24 hours. After the solution had been cooled to 60° C., it was poured into 250 ml of ice water, precipitate being obtained. The acidic suspension was neutralized with 167 ml of concentrated ammonia solution and then filtered. The dark brown residue was washed with water and dried under reduced pressure at 75° C. (3.9 g). The crude product was purified by precipitation in sulfuric acid.

Yield: 1.42 g of brownish microcrystals

SIMS: [M+H]⁺=526.1

UV/Vis: λₘₐₓ=385 (S), 363, 345 nm in formic acid,

λ_{fluor.}=430 nm in formic acid

### Example 7

### Copper cyclo-2,4′:2′,7′:2″,4′″:2′″,7-quaterbenzimidazole

0.465 g (1.0 mmol) of cycloquaterbenzimidazole was suspended in 100 ml of dimethylformamide, and 0.200g (1.0 mmol) of anhydrous copper(II) acetate was added. The reaction solution was refluxed for 20 hours with stirring and under nitrogen. The suspension was cooled and then filtered, and the residue was washed with dimethylformamide and water and dried at 60° C. under reduced pressure.

Yield: 0.223 g of brownish powder

### Example 8

### Nickel cyclo-2,4′:2′,7″:2″,4′″:2′″,7-quaterbenzimidazole

4.4 g (26 mmol) of ammonium 2,3-diaminobenzoate and 0.842 g (6.5 mmol) of anhydrous nickel chloride were introduced into 120g of 85% strength polyphosphoric acid at 100° C. with stirring and under nitrogen. Thereafter, the reaction solution was heated to 180° C. with evolution of ammonia and kept at this temperature for 24 hours. After the solution had been cooled to 60° C., it was poured into 250 ml of ice water, a precipitate being obtained. The acidic suspension was brought to pH 8.0 with 175 ml of concentrated ammonia solution and then filtered. The dark brown residue was washed with water and dried under reduced pressure at 75° C. (3.30 g). For purification, the crude product was dissolved in 40 ml of formic acid at 80° C. The solution was filtered, and 40 ml of n-propanol were added. After stirring for two hours at 80° C., the solution was allowed to cool to room temperature, during which a precipitate was obtained. The solid was separated off, washed with a solution of formic acid and n-propanol (1:1) and dried at 60° C. under reduced pressure.

Yield: 0.94 g of yellow-green microcrystals

SIMS: [M+H]⁺=521.2

UV/Vis: λₘₐₓ=415 (S), 390 (S), 361 nm in formic acid,

λ_{fluor.}=470 nm in formic acid

### Example 9

### Magnesium cyclo-2,4″:2′,7″:2″,4′″:2′″,7-quaterbenzimidazole

0.465 g (1.0 mmol) of cycloquaterbenzimidazole was suspended in 50 ml of dimethylformamide, and 0.322 g (1.5 mmol) of magnesium acetate tetrahydrate was added. The reaction solution was refluxed for 20 hours with stirring and under nitrogen. The suspension was cooled and then filtered, and the residue was washed with dimethylformamide and water and dried at 60° C. under reduced pressure.

Yield: 0.506 g of yellowish microcrystals

MALDI-MS: [M+H]⁺=487.3

UV/Vis: λₘₐₓ=405 (S), 385, 352 nm in formic acid,

λ_{fluor.}=492 nm in formic acid

### Example 10

Platinum cyclo-2,4′:2′,7″:2″,4′″:2′″7-quaterbenzimidazole

2.32 g (5.0 mmol) of cycloquaterbenzimidazole were dissolved in a solution of 200 ml of acetic acid and 40 ml of water. After the addition of 0.82 g (10 mmol) of sodium acetate, the solution was stirred for 10 minutes and then 2.18 g (5.25 mmol) of potassium tetrachloroplatinate were added. The reaction solution was heated to 85° C. and kept at this temperature for 24 hours. After cooling to room temperature, the solid was filtered off with suction, washed with dilute acetic acid and water and dried at 75° C. under reduced pressure.

Yield: 3.58 g of black-brown microcrystals

MALDI-MS: [M+H]⁺=657.1

### Example 11

### Platinum cyclo-2,4′:2′,7″(4″):2″,4′″(7′″):2′″,7″″(4″″):2″″,4(7)-quinquebenz-imidazole

0.580g (1.00 mmol) of cycloquinquebenzimidazole were dissolved in a solution of 50 ml of acetic acid and 10 ml of water. After the addition of 0.164 g (2.00 mmol) of sodium acetate, the solution was stirred for 10 minutes and then 0.457 g (1.10 mmol) of potassium tetrachloroplatinate was added. The reaction solution was heated to 85° C. and kept at this temperature for 44 hours. After cooling to room temperature, the solid was filtered off with suction, washed with dilute acetic acid and water and dried at 75° C. under reduced pressure.

Yield: 0.790g of black-brown microcrystals

MALDI-MS: [M+H]⁺=773.2

### Example 12

### Cyclo-2,7′:2′,7″:2″,7′″:2′″,7-quaterbenzoxazole

3.90 g (22.9 mmol) of ammonium-3-amino-2-hydroxybenzoate were introduced in portions into 93.6 g of 85% strength polyphosphoric acid with stirring and under nitrogen. Thereafter, the reaction solution was heated to 160° C. and kept at this temperature for 24 hours. After the solution had been cooled to 85° C., it was brought into ice water, a precipitate being obtained. The precipitate was filtered off with suction and washed with water. The filter residue was suspended in water, and the solution was neutralized with ammonia solution. The solid was filtered off with suction, washed with water and dried at 60° C. under reduced pressure (1.86 g). For purification, the solid was dissolved in concentrated sulfuric acid and precipitated by slow addition of water.

Yield: 0.69 a (25%) of yellowish microcrystals

| C₂₈H₁₂N₄O₄•H₂O | Calc. | C 69.14 | H 2.90 | N 11.52 | O 16.45 |
|---|---|---|---|---|---|
| M = 468.43 + 18.02 = | Found | C 68.4 | H 3.1 | N 11.1 | O 16.4 |
| 486.44 | | | | | |

### Example 13

### 2-Acetamido-4-methylphenol (a)

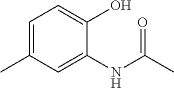

12.3 g (100 mmol) of 2-amino-4-methylphenol were dissolved in 100 ml of absolute THF. 10.4 g (102 mmol) of acetic anhydride were added to the brown solution at room temperature. The temperature increases to 45° C., and stirring was carried out for five hours without further heating.

The solvent was removed under reduced pressure, and the solid was dried at 60° C. under reduced pressure. The yield of gray powder is quantitative.

¹H-NMR 360 MHz, d6-DMSO δ=9.50-9.45 (b 1H, NH/OH), 9.22-9.18 (b, 1H, NH/OH) 7.50 (b, 1H, CH), 6.80 (b, 2H, CH), 2.17 (s, 3H, CH₃), 2.02 (s, 3H, CH₃) ppm.

¹³C-NMR 80 MHz, d6-DMSO δ=168.9 (HNCO), 145.5 (C—O), 126.0, 127.4 (C—CH₃, C—N), 125.0, 122.7, 115.7 (CH, CH, CH), 23.5 (CH₃), 20.3 (CH₃) ppm.

Rf(CHCl₃: acetic acid=20:5)=0.6

m.p.: 170-173° C.

### 2-Carboxy-4-methylbenzoxazolidinone (b)

50g of dry potassium carbonate were finely milled in a coffee mill, after which 5.0 g of (a) (30 mmol) were milled with the potassium carbonate within three times 20 seconds in the coffee mill. The white mixture was rolled in a 250 ml can with 5 steel balls (diameter 3 cm) on a roller stand. The balls were separated off and the mixture was milled for 60 seconds in the coffee mill before it was introduced into a dry autoclave. This was evacuated for five minutes before 50 bar CO₂ was forced in and heated to 220° C. CO₂ consumed in the heat-up phase was replenished. After 220° C. had been reached, the pressure was increased to 100 bar and the system was kept at this temperature for 14 hours. After cooling of the autoclave, a white lump was removed and is milled in a coffee mill. The crude product in the presence of excess K₂CO₃ was characterized as follows:

¹H-NMR, 360 MHz, D₂O δ=7.18 (s, 1H, CH), 7.02 (s, 1H, CH), 2.28 (s, 3H, CH₃), 1.90 (s, 3H, CH₃) ppm.

¹³C-NMR, 90 MHz, D₂O δ=184.2 (CO), 175.9 (CO), 146.5, 145.9, 135.0, 123.6 (C—OH, C—N, C—CH₃, C—CO₂H), 123.6 (CH), 188.8 (CH), 26.3 (CH₃), 23.3 (CH₃) ppm.

R_{f} (chloroform:acetic acid: methanol=25:5:5)=0.8

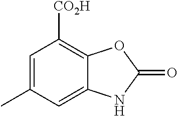

5.0 g (2.7 mmol) of the above-described mixture of K₂CO₃ and crude product were stirred in 5 ml of water and brought to a pH of 1 by adding a little concentrated HCl. The suspension was stirred for 44 hours at 50° C., and the precipitate formed was cooled, left to stand overnight and then filtered off. It was washed neutral with a small amount of ice water. After drying under reduced pressure at 80° C., 0.15 g (41% over three stages) of a white powder was obtained.

¹H-NMR, 360 MHz, d6-DMSO, δ=11.9 (bs, 1H, NH), 7.30 (s, 1H, CH), 7.03 (s, 1H, CH), 2.02 (s, 3H, CH₃) ppm.

¹³C-NMR, 80 MHz, d6-DMSO, δ=164.7 (CO₂H), 154.5 (NC═OO), 141.0 (C—CO₂H), 132.9, 131.5, 114.1, (C—N, C—O, C—CH₃), 123.0, 114.1 (CH, CH), 20.6 (CH₃) ppm.

R_{f} (toluene:ethanol:acetic acid=25:10:1)=0.6

A peak at 193g/mol (molecular ion peak) is detected in the mass spectrum.

HRMS (ESI): 192.0296 (found)

192.0297 (calc.)

m.p.: 283-286° C.

### Disodium 3-amino-5-methylsalicylate (c)

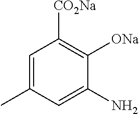

1.01 g (5.23 mmol) of (b) were stirred in 10 ml of water, and 0.63 g (15.7 mmol) of sodium hydroxide was added. The red-brown solution was stirred under reflux for 16 hours. A further 0.21 g (5.2 mmol) of sodium hydroxide was then added. The water was evaporated at 80° C. under reduced pressure and a brown powder which contained 3 eq of sodium hydroxide was obtained.

¹H-NMR, 360 MHz, D₂O, δ=7.15 (s, 1H, CH), 6.90 (s, 1H, CH), 2.17 (s, 3H, CH₃) ppm;

the NH, resonance was not observed.

¹³C-NMR, 90 MHz, D₂O/d6-dmso 1:1 δ=175.7 (CO₂H), 148.9 (C—OH), 136.8 (C—NH₂(C—CH₃), 128.0 (C—NH₂/C—CH₃), 121.2 (CH), 120.4 (CH), 119.9 (C—CO₂H), 22.3 (CH₃) ppm.

R_{f}(CHCl₃:methanol:acetic acid=20:5:1)=0.7

HRMS (ESI): 166.0504 (found)

166.0504 (calc.) corresponds to doubly protonated molecule C₈H₈NO₃

### Tetramethylcycloquaterbenzoxazole (d)

### (2,6,10,14-Tetramethylcyclo-2,7′:2′,7″:2″,7′″:2′″,7-quaterbenzoxazole)

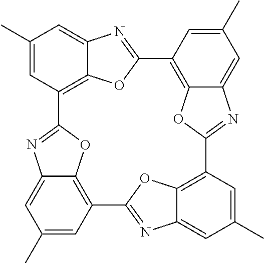

228g of polyphosphoric acid were heated to 180° C. under a nitrogen atmosphere. 5.7 g (18 mmol) of c, which still contained 2 equivalents of NaOH, were added as a solid in the course of 30 minutes. The reaction mixture was stirred for 24 hours at this temperature before it was introduced into 300 ml of water at 90° C. A further 150 ml of water and 200 ml of n-butylglycol were added to the gray suspension and heating was effected for 1.5 hours to 100° C. The suspension was filtered with suction while hot. The pressed cake was washed neutral with warm water and then boiled for three hours in 100 ml of 1M NaOH. The suspension was filtered again, washed with water and acetone and dried. Boiling was effected again in 50 ml of n-butylglycol for three hours at 150° C. Thereafter, filtration was effected and the residue was washed with acetone and dried at 140° C. 1.05 g (44%) of a gray powder were obtained. This was stirred in 20g of tetrachloronaphthalene for 7 hours at 225° C. before dilution was effected with 20 ml of NMP and the solid was filtered off at 100° C. After washing with acetone, 0.91 g (38%) of a pale powder was obtained. This material was stirred in 30g of phenol at 200° C. for 5.5 hours, filtered with suction at 100° C. and washed with ethanol, acetone and methanol. 0.73 g (30%) of a pale gray powder was obtained. A transmission electron micrograph showed particles having a size of about 50 nm.

calc.

C, 71.4; (found), 73.3 (calc.); H, 4.0; (found) 3.8 (calc.); N, 9.9; (found) 10.7 (calc.); O, 13.8; (found) 12.2 (calc.).

### Example 14

### 5-tert-Octylsalicylic acid (a)

80g (0.39 mol) of sodium were melted in 1200 ml of anhydrous xylene, and 80g (0.39 mol) of tert-octylphenol were slowly introduced. In addition, 20 ml of THF were added in order to improve the stirrability of the mixture. After all the sodium had been consumed, a further 8.93 g (0.39 mol) of sodium were added. CO₂ was passed through the reaction mixture under reflux for 20 hours. By adding 250 ml of water, residues of unconverted sodium were hydrolyzed. The phases were separated and the organic phase was extracted with 3 times 50 ml of 2M NaOH. The combined aqueous phases were acidified with concentrated HCl, and the precipitate was filtered off and washed neutral. The precipitate was taken up in 100 ml of aqueous NaHCO₃ solution and 100 ml of toluene, the phases were separated and the aqueous phase was washed repeatedly with 50 ml of toluene each time. The aqueous phase was acidified with concentrated HCl, and the precipitate was filtered off, washed and dried under reduced pressure. 47.6 g (48.8%)

¹H-NMR (CDCl₃): δ=10.4-10.3 (bs, 1H, OH), 7.9 (s, 1H, Ph-H), 7.5 (d, 1H, Ph-H), 6.9 (s, 1H, Ph-H), 1.7 (s, 2H, CH₂), 1.35 (s, 6H, CH₃), 0.7 (s, 9H, CH₃) ppm.

### Methyl 5-tert-octylsalicylate (b)

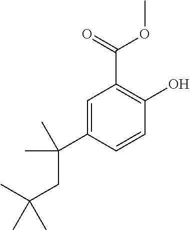

50 ml of anhydrous methanol and 4.0 g (40.8 mmol) were boiled together with 46.3 g (185 mmol) of 3-tert-octylsalicylic acid for 128 hours. Thereafter, the excess methanol was removed in a rotary evaporator and the residue was taken up in a mixture of 100 ml of ethyl acetate and 75 ml of NaHCO₃ (caution: foam). The phases were separated and the organic phase was washed with 3 times 30 ml of NaHCO₃. The organic phase was dried over MgSO₄ and the solvent was removed in a rotary evaporator. 45.0 g (92%) of a white solid were obtained. Rf_{(toluene:ethanol=10:2)}=0.9

¹H-NMR (CDCl₃)=10.7-10.6 (bs, 1H, OH), 7.8 (s, 1H, Ph-H), 7.5 (d, 1H, Ph-H), 6.9 (d, 1H, Ph-H), 4.0 (s, 3H, OCH₃), 1.80 (s, 2H, CH₂), 1.25 (s, 6H, CH₃), 0.78 (s, 9H, CH₃) ppm.

### Methyl 3-nitro-5-tert-octylsalicylate (c)

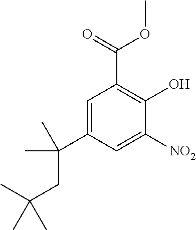

43.62 g (0.165 mmol) of the methyl ester (b), together with 0.1 g of sodium nitrite, were dissolved in 284g of 80% strength H₂SO₄, and 10.4 g (165 mmol) of fuming nitric acid were added in the course of 90 minutes at from 5 to 10° C. Stirring was effected for 6.5 hours at this temperature before the mixture was carefully poured onto ice water. The aqueous phase was extracted with 3 times 100 ml of diethyl ether, the combined organic phases were washed with 50 ml of NaHCO₃ solution and dried over MgSO₄ and the solvent was removed from the rotary evaporator. After chromatography (petroleum ether/ethyl acetate=9:1) 51.1 g (65%) of the product were obtained. M.p. 60° C.

¹H-NMR (DMSO) δ=11.2 (bs, 1H, OH), 8.15 (s, 1H, Ph-H), 8.0 (s, 1H, Ph-H), 3.9 (s, 3H, OCH₃), 1.70 (s, 2H, CH₂), 1.25 (s, 6H, CH₃), 0.65 (s, 9H, CH₃) ppm.

¹³C-NMR (DMSO, DEPT) δ=167.8 (CO₂Me), 150.2 (PhC—OH), 140.8 (PhC-tertOctyl), 138.5 (PhC—NO₂), 131.8 (PhCH), 127.7 (PhCH), 116.3 (PhC—CO₂Me), 55.5 (CH₂), 53.0 (OCH₃), 37.9 (CCH₃), 31.9, 31.5, 30.8 (CH₃) ppm.

### 3-Nitro-5-tert-octylsalicylamide (d)

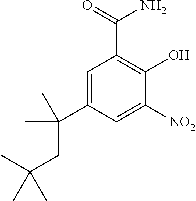

27.45 g (80 mmol) of the ester (c) were dissolved in 100 ml of THF, and 150 ml of 1 M NH₃ solution (150 mmol) were added. With the aid of a gas pocket, an NH₃ atmosphere of 1 bar was maintained. Stirring was effected for 5 weeks at room temperature under these conditions until complete conversion was achieved. The solvent was then removed under reduced pressure. The residue was stirred with 300 ml of petroleum ether, filtered off and dried under reduced pressure. 24.8 g (quantitative) of an orange powder were obtained.

¹H-NMR (CDCl₃) δ=12.0-11.0 (b, 1H, OH), 8.7 (s, 1H, Ph-H), 8.25 (s, 1H, Ph-H), 7.9-7.8 (bs, 1H, NH), 6.6-6.5 (bs, 1H, NH), 1.8 (s, 2H, CH₂), 1.4 (s, 6H, CH₃), 0.7 (s, 9H, CH₃) ppm.

¹³C-NMR (CDCl₃) δ=165.6 (CONH₂), 151.6 (PhC—OH), 142.9 (C-tert-octyl), 138.7 (PhCH), 134.0 (C—NO₂), 126.2 (PhCH), 121.8 (PhC—CONH₂), 56.3 (CH₂), 38.5 (CH₃), 32.4, 31.9, 31.2 (CH₃) ppm.

### 3-Amino-5-tert-octylsalicylamide (e)

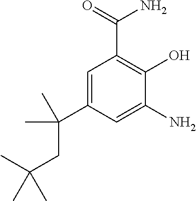

19.7 g (67 mmol) of the nitro compound (d) were dissolved in 100 ml of methanol at room temperature. 2.0 g of palladium on active carbon (10% of Pd) were added and hydrogen was passed in at atmospheric pressure in the course of 24 hours. The catalyst was filtered off with suction and the methanol was removed under reduced pressure. After washing with petroleum ether, filtration and drying of the solid, 15.8 g (89%) of a solid were obtained.

¹H-NMR (DMSO) δ=8.3 (bs, 1H, CONH₂), 7.8 (bs, 1H, CONH₂), 7.1 (s, 1H, Ph-H), 6.9 (s, 1H, Ph-H), 1.6 (s, 2H, CH₂) 1.2 (s, 6H, CH₃), 0.8 (s, 9H, CH₃) ppm. OH and NH not observed.

### Tetra-tert-octylcycloquaterbenzoxazole (f)

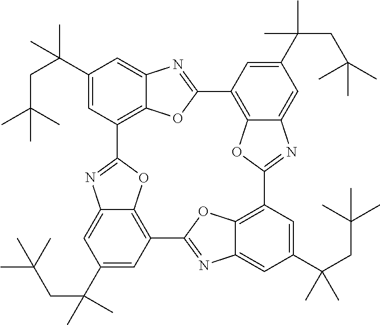

15g of polyphosphoric acid were heated to 180° C. 200 mg (0.8 mmol) of the compound (e) were added and stirring was effected at this temperature for 24 hours. Thereafter, the solution was added to ice water and the resulting precipitate was filtered off. 120 mg of a black solid were obtained. By means of mass spectroscopy (MALDI-TOF-SIMS), it was possible to detect the molecular ion peak at 917.3 dalton.

### Example 15

### Cycloquaterbenzoxazole

### (Cyclo-2,4′:2′,4″:2″,4′″:2′″,4-quaterbenzoxazole)

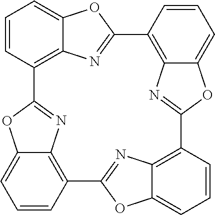

2.00 g (13 mmol) of 3-hydroxyanthranilic acid (Aldrich) are slowly introduced into 100 ml of polyphosphoric acid at 180° C. After 18 hours, the solution is added to 250 ml of water at 80° C. The precipitate is filtered off with suction and is washed neutral with warm water.

980 mg (65%) of an ochre precipitate, which remains as an undecomposed solid up to 350° C., are obtained.

In the mass spectrum, the molecular ion peak of the cyclotetramer is detected at 469.09 g/mol, the molecular ion peak of the cyclopentamer is detected at 586.40 g/mol. UV spectrum from H₂SO₄ λₘₐₓ=262 nm (52 l/cm·g), λₘₐₓ=338 nm (93 l/cm·g).

### Example 16

### 3,4-Diamino-2-naphtboic acid (a)

Azo coupling: 8.74 g (50.0 mmol) of 99% strength sulfanilic acid were dissolved in 100 ml of water with stirring by addition of 3.4 g 50% strength sodium hydroxide solution. After the addition of a solution of 3.46 g (50.0 mmol) of sodium nitrite in water, the reaction solution was filtered and then slowly metered into a solution of 50g of ice, 50 ml of water and 14.7 g of concentrated hydrochloric acid. After stirring for 2 hours at from 0 to 5° C., the excess nitrite was destroyed with 2g of amidosulfonic acid. The diazonium salt solution thus obtained was metered into a solution of 11.0 g (50.0 mmol) of 85% strength 3-amino-2-naphthoic acid and 17.85 g (220 mmol) of sodium carbonate in 500 ml of water, the pH being kept above 8 by addition of 3g of 50% NaOH. The azo dye solution was stirred for a further hour.

Reduction: 2.86 g of 50% NaOH were added (pH 8.9) to the solution of the azo dye and heating was effected under nitrogen to 80 to 90° C. 23.62 g (136 mmol) of sodium dithionite were added in portions, the pH of the solution being kept at about 9 by addition of 50% NaOH. After decolorization of the reaction batch, the latter was cooled to room temperature.

### Cyclo-2,4′:2′,9″:2″,4′″:2′″,9-quaternaphtho[1,2-d]imidazole/

### Cyclo-2,4′:2′,9″:2″,4′″:2′″,9″″:2″″,4-quinquenaphtho[1,2-d]imidazole (b)

5.00 g (24.9 mmol) of 3,4-diamino-2-naphthoic acid were dissolved in 120g of 85% strength polyphosphoric acid with stirring and under nitrogen. The reaction solution was heated to 150° C. and kept at this temperature for 24 hours. After the solution had been cooled to 60° C., it was poured into ice water, a precipitate being obtained. The precipitate was filtered off with suction and washed with water. The moist filter residue was stirred in hot water. The suspension was made alkaline (pH 12) with ammonia solution, stirred overnight, brought to pH 8-9 with HCl and filtered. The solid was washed with water and dried under reduced pressure at 70° C. 3.73 g of brown crude product were obtained. For purification, the crude product was suspended in 15.7 g of N-methylpyrrolidone (NMP) and heated to 110° C., a part of the solid going into solution. The suspension was cooled to room temperature and filtered over a Blauband filter. The residue was washed with four times 5 ml of NMP. (The filtrate was worked up to give cycloquinquenaphthimidazole (see further below).) The filter residue was suspended in 50 ml of methanol, filtered, washed with methanol and dried at 40° C. under reduced pressure (0.90 g). The yellowish solid was suspended again in 42.7 g of NMP. The suspension was heated to the boil, cooled to 80° C. and filtered over a Blauband filter. The residue was washed with 6 ml of NMP and ethanol and dried at 80° C. under reduced pressure.

Yield: 0.82 g (19%) of yellowish microcrystals

| C₄₄H₂₄N₈•1.5 H₂0 | Calc. | C 76.40 | H 3.93 | N 16.20 |
|---|---|---|---|---|
| M = 664.73 + 1.5 × 18.02 = | Found | C 76.8 | H 4.2 | N 16.2 |
| 691.75 | | | | |

MALDI-MS: [M+H]⁺=665.2

The NMP-containing filtrate was evaporated down at 140° C. under reduced pressure from a waterjet pump to give a paste, taken up in 160 ml of methanol and refluxed for 4 hours. The suspension was cooled to 40° C., filtered, washed with four times 16 ml of methanol in each case and dried at 80° C. in a through-circulation drying oven (1.68 g). For purification, the solid was introduced into 58.7 g of 95.4% strength sulfuric acid in the course of 30 minutes and was dissolved after stirring for 3 hours at 25° C. 53.7 g of 50% strength sulfuric acid were metered into the solution in the course of 7.5 hours, the resulting precipitate being filtered off with suction over a G4 glass frit and washed four times with a total of 23 ml of 50% strength sulfuric acid. The filter residue was suspended in 110 ml of water and stirred for one hour. The solid was filtered off with suction, washed with hot water and dried at 80° C. in a through-circulation drying oven (1.10 g). The solid was refluxed in 85 ml of ethanol for one hour and filtered while hot. The filtrate was evaporated to dryness.

Yield: 0.034 g (0.5%) of yellowish powder

According to elemental analysis, the macrocycle crystallized with four mole equivalents of ethanol.

| | | | | |
|---|---|---|---|---|
| C₅₅H₃₀N₁₀•4 C₂H₅OH | Calc. | C 74.54 | H 5.36 | N 13.80 |
| M = 830.91 + 4 × 46.07 = 1015.18 | Found. | C 74.5 | H 5.1 | N 13.3 |

MALDI-MS: [M+H]⁺=831.3

### Example 17

### Cyclo-2,9′:2′,9″:2″,9′″:2′″,9-quaternaphthol[1,2-d]oxazole

15.5 g (76.3 mmol) of 4-amino-3-hydroxy-2-naphthoic acid were introduced in portions into 372g of 85% strength polyphosphoric acid. The reaction solution was heated to 160° C. and kept at this temperature for 24 hours. After the solution had been cooled to 85° C., it was poured into ice water with stirring, a precipitate being obtained. The precipitate was filtered off with suction and washed with water until the wash water had a neutral pH. The crude product was dried at 60° C. under reduced pressure (10.4 g). For purification, the crude product was dissolved in 375g of 95.4% strength sulfuric acid. The solution was filtered over a glass frit, and 111g of 50% strength sulfuric acid were added in the course of four hours, crystals forming. The solid was filtered off with suction over a G4 glass frit and in each case washed four times with 85% strength and 50% strength sulfuric acid. The residue was stirred in hot water. The suspension was filtered over a Blauband filter. The residue was washed pH-neutral with hot water and dried at 80° C. in a through-circulation drying oven.

Yield: 4.56 g (36%) of yellowish microcrystals; decomposition >620° C.

| | | | | | |
|---|---|---|---|---|---|
| C₄₄H₂₀N₈O₄ | Calc. | C 79.04 | H 3.01 | N 8.38 | O 9.57 |
| M = 668.67 | Found | C 79.1 | H 3.1 | N 8.4 | O 9.6 |

MALDI-MS: M⁺=667.9

UV/Vis: λₘₐₓ (1 g ε)=420 (S), 404 (4.96), 326 (4.69), 268 (4.89), 220 nm (4.71) in sulfuric acid, λ_{fluor.}=484 nm in sulfuric acid

### Example 18

### 4-Amino-3-hydroxy-2-naphthoic acid

A solution of 35.00 g (200 mmol) of sodium dithionite in 200 ml of water was added dropwise to a solution of 10.61 g (25.0 mmol) of calcium 3-hydroxy-2-naphthoate-1-azo-2″-(5″-methylbenzenesulfonate) (Lithol Ruby Red) in 500 ml of water and 50g of 50% NaOH at 80° C., the pH decreasing to 12.0. The reaction solution was refluxed for two hours and then cooled to 60° C. and filtered. After the filtrate had been brought to pH 4.5 with concentrated HCl, the solid was filtered off with suction, washed with water and dried over sodium hydroxide in a desiccator.

Yield: 3.14 g (<62%) of yellowish powder; m.p. 231-234° C. (Ref.: 241° C. (decomposition))

### Example 19

### Sulfonation of cyclo-2,9′:2′,9″:2″,9′″:2′″,9-quaternaphtho[1,2-d]oxazole

5.00 g (7.5 mmol) of cyclo-2,9′:2′,9″:2″,9′″:2′″,9-quaternaphtho[1,2-d]oxazole were dissolved in 75g of 6% strength oleum with stirring. The solution was heated to 150° C. and stirred for two hours at this temperature. Thereafter, the reaction solution was cooled to room temperature and precipitated on a little ice. The suspension was diluted with 2 l of acetone, a finely crystalline precipitate forming. The precipitate was filtered off with suction over a glass frit. The filter residue was suspended in acetone, filtered off with suction, washed with acetone and dried at 60° C. under reduced pressure.

Yield: 6.8 g of yellow microcrystals

According to elemental analysis and the mass spectrum, substantially a tetrasulfonated product was present.

C₄₄H₂₀N₄O₁₆S₄×10 H₂0 Calc. C, 45.20; H, 3.45; N, 4.79; O, 35.58; S, 10.97.

M=988.90+10×18.02=1169.05 Found C, 46.4; H, 3.8; N, 4.7; O, 35.4; S, 10.3.

MS (ESI): [M−4H]⁴⁻=245.99, [M−3H]³⁻=328.32, [M−2H]²⁻=492.98

### Example 20

### Sulfonation of cyclo-2,9′:2′,9″:2″,9′″,2′″,9-quaternaphtho[1,2-d]oxazole

2.50 g (3.75 mmol) of cyclo-2,9′:2′,9″:2″,9′″:2′″,9-quaternaphtho[1,2-d]oxazole were dissolved in 37.5 g of 6% strength oleum with stirring. The solution was heated to 60° C. and stirred for 16 hours at this temperature. Thereafter, the reaction solution was cooled to room temperature and precipitated on 60 g of ice. The suspension was diluted with acetone, a finely crystalline precipitate forming. The precipitate was filtered off with suction over a glass frit. The filter residue was suspended in acetone, filtered off with suction, washed with acetone and dried at 60° C. under reduced pressure.

Yield: 1.95 g of yellow microcrystals

From the elemental analysis for nitrogen (5.6%) and sulfur (9.6%), a degree of sulfonation of three was calculated.

### Example 21

### Sulfonation of cyclo-2,9′:2′,9″:2″,9′″:2′″,9-quaternaphtho[1,2-d]oxazole

2.50 g (3.75 mmol) of cyclo-2,9′:2′,9″:2″,9′″:2′″,9-quaternaphtho[1,2-d]oxazole were dissolved in 37.5 g of 6% strength oleum with stirring. The solution was heated to 60° C. and stirred for two hours at this temperature. Thereafter, the reaction solution was cooled to room temperature and precipitated on 60 g of ice. The suspension was diluted with acetone, a finely crystalline precipitate forming. The precipitate was filtered off with suction over a glass frit. The filter residue was suspended in acetone, filtered off with suction, washed with acetone and dried at 60° C. under reduced pressure.

From the elemental analysis for nitrogen (6.3%) and sulfur (4.8%), a degree of sulfonation of 1.3 was calculated.

### Example 22

### Chlorination of cyclo-2,9′:2′,9″:2″,9′″:2′″,9-quaternaphtho[1,2-d]oxazole

30 g of aluminum chloride, 6.76 g of sodium chloride and 0.22 g of sodium bromide were heated together to 160° C. The resulting melt was cooled to 100° C., and 6.68 g (10.0 mmol) of cyclo-2,9′:2′,9″:2″,9′″:2′″,9-quaternaphtho[1,2-d]oxazole were added in portions while passing in chlorine. After addition of the macrocycle, the temperature of the reaction mixture was increased to 140-150° C., chlorine (18.7 g altogether) being passed in for five hours. The melt was then heated to 180° C. and kept at 180° C. for 30 minutes. The melt was precipitated on water. The resulting solid was filtered off with suction, washed pH-neutral and salt-free with water and dried at 60° C. under reduced pressure (10.0 g). For purification, the crude product was dissolved in 250 g of 97% strength sulfuric acid at 50-55° C. At 40-45° C., 86 g of 50% strength sulfuric acid were added dropwise. After the suspension had been cooled to room temperature, it was filtered over a glass frit (P4). The crystals were washed with 85% strength and then with 50% strength sulfuric acid, suspended in water, filtered off with suction, washed pH-neutral and dried at 60° C. under reduced pressure.

Yield: 8.8 g of yellow microcrystals

According to elemental analysis, the degree of chlorination n was 8.2.

| | | | | | |
|---|---|---|---|---|---|
| C₄₄H₂₀₋ₙClₙO₄N₄ | Found | C 54.3 | H 1.5 | Cl 29.9 | N 5.8 |

According to the mass spectrum (SIMS), substantially a macrocycle mixture having five to nine chlorine atoms was present (main peak with eight chlorine atoms at M⁺=944.9).

### Example 23

### α-Amidomethylation of cyclo-2,9′:2′,9″:2″,9′″:2′″,9-quaternaphtho[1,2-d]oxazole with N-hydroxymethylpyrrolidone

2.50 g (3.73 mmol) of cyclo-2,9′:2′,9″:2″,9′″:2′″,9-quaternaphtho[1,2-d]oxazole (CQNO) and 2.58 g (22.4 mmol) of N-hydroxymethylpyrrolidone were dissolved in 50 ml of concentrated sulfuric acid and stirred for 20 hours at 70° C. After cooling to room temperature, the solution was precipitated on an ice-water mixture. The precipitate was filtered off with suction and washed pH neutral with water. The filter residue was suspended in water and brought to pH 9 with ammonia solution. The solid was filtered off with suction, washed with water and dried at 60° C. under reduced pressure.

Yield: 2.60 g of yellow powder

### Example 24

### α-Amidomethylation of cyclo-2,9′:2′,9″:2″,9′″:2′″,9-quaternaphtho[1,2-d]oxazole with caprolactam/formaldehyde

3.34 g (5.0 mmol) of cyclo-2,9′:2′,9″:2″,9′″:2′″,9-quaternaphtho[1,2-d]oxazole (CQNO), 0.9 g (30 mmol) of paraformaldehyde and 3.4 g (30 mmol) of caprolactam were dissolved in succession with stirring in 100 g of 85% strength polyphosphoric acid at 40-45° C. The reaction mixture was heated to 105° C. and stirred at this temperature for 8.5 hours. After cooling to 90° C., the reaction solution was precipitated on ice/water and brought to pH 8.5 with concentrated ammonia solution while cooling (addition of ice). The solid was filtered off with suction, washed salt-free with water and dried at 60° C. under reduced pressure.

Yield: 4.9 g of yellow powder

According to the MALDI mass spectrum, the product mixture, which is soluble in acetic acid, contained, in addition to unknown compounds, CQNO having one to five N-methylene caprolactam radicals ([M+H]⁺=794.2, 919.3, 1044.4, 1169.4, 1294.6).

### Example 25

### α-Amidomethylation of cyclo-2,9′:2′,9″:2″,9′″:2′″,9-quaternaphtho[1,2-d]oxazole with N-hydroxymethyl-5-tert-butylcaprolactam

3.34 g (5.0 mmol) of cyclo-2,9′:2′,9″:2″,9′″:2′″,9-quaternaphtho[1,2-d]oxazole (CQNO) and 5.97 g (30 mmol) of 5-tert-butylcaprolactam were dissolved in succession with stirring in 107 g of 85% strength polyphosphoric acid at 40-45° C. The reaction mixture was heated to 105° C. and stirred at this temperature for 8 hours. After cooling to 90° C., the reaction solution was precipitated on ice/water and brought to pH 8.5 with concentrated ammonia solution while cooling (addition of ice). The solid was filtered off with suction, washed salt-free with water and dried at 60° C. under reduced pressure.

Yield: 7.2 g of yellow powder

The product mixture was soluble in tetrahydrofuran (THF).

UV/Vis: λₘₐₓ=400, 324, 270, 220 nm in sulfuric acid,

λₘₐₓ=370, 320, 266 nm in THF

### Example 26

### Imidazolylmethylation of cyclo-2,9′:2′,9″:2″,9′″:2′″,9-quaternaphtho[1,2-d]oxazole with imidazole/formaldehyde

1.34 g (44.8 mmol) of paraformaldehyde and 3.06 g (45.0 mmol) of imidazole were introduced simultaneously into 75 ml of 97% strength sulfuric acid with stirring at room temperature, whereby the reaction temperature should not exceed 50° C. The reaction solution was then heated to 80° C. and stirred at this temperature for 70 minutes. As soon as formaldehyde was no longer detectable, 5.00 g (7.47 mmol) of cyclo-2,9′:2′,9″:2″,9′″:2′″,9-quaternaphtho[1,2-d]oxazole (CQNO) were added at 58-60° C. in the course of one hour. The solution was stirred for 165 minutes at 60° C., then heated to 100° C. and kept at this temperature for three hours. After cooling to room temperature, the solution was precipitated on an ice-water mixture and brought to pH 9 with ammonia solution, the temperature being kept below 20° C. by adding ice. The solid was filtered with suction, washed pH-neutral and salt-free with water and dried at 60° C. under reduced pressure.

Yield: 6.5 g of yellow powder

UV/Vis: λₘₐₓ=406, 326, 270, 220 nm in sulfuric acid,

λₘₐₓ=368, 320, 270 nm in acetic acid

According to MALDI-MS, a product mixture comprising CQNO with n=1-6 methyleneimidazole radicals was present (highest peak at 909.3 of CQNO with n=3).

### Example 27

### Imidazolylmethylation of cyclo-2,9′:2′,9″:2″,9′″:2′″,9-quaternaphtho[1,2-d]oxazole with N-methylimidazole/formaldehyde

1.34 g (44.8 mmol) of paraformaldehyde and 3.69 g (45.0 mmol) of N-methylimidazole were introduced and added dropwise, respectively, simultaneously into 75 ml of 97% strength sulfuric acid with stirring at room temperature, whereby the reaction temperature should not exceed 50° C. The reaction solution was then heated to 80° C. and stirred at this temperature for 70 minutes. As soon as formaldehyde was no longer detectable, 5.00 g (7.47 mmol) of cyclo-2,9′:2′,9″:2″,9′″:2′″,9-quaternaphtho[1,2-d]oxazole (CQNO) were added at 58-60° C. in the course of one hour. The solution was stirred for 165 minutes at 60° C., then heated to 100° C. and kept at this temperature for three hours. After cooling to room temperature, the solution was precipitated on an ice-water mixture and brought to pH 9 with ammonia solution, the temperature being kept below 20° C. by adding ice. The solid was filtered with suction, washed pH-neutral and salt-free with water and dried at 60° C. under reduced pressure.

Yield: 7.0 g of yellow powder

UV/Vis: λₘₐₓ=368, 320, 270 nm in acetic acid

According to MALDI-MS, a product mixture comprising CQNO with n=1-5 methylene-N-methylimidazole radicals was present (highest peak at 951.3 of CQNO with n=3).

### Example 28

### 4-Amino-7-bromo-3-hydroxy-2-naphthoic acid

Azocoupling: 4.37 g (25.0 mmol) of 99% strength sulfanilic acid were dissolved in 70 ml of water with stirring by adding 2.09 g of 50% strength sodium hydroxide solution. After the addition of a solution of 1.73 g (25.0 mmol) of sodium nitrite in water, the reaction solution was tested for nitrite (negative), a further 0.33 g (4.8 mmol) of sodium nitrite was added and the reaction solution was then added slowly to a mixture of 100 g of ice/water and 9.70 g (98.4 mmol) of concentrated HCl. After stirring for 2 hours at 0-5° C., the excess nitrite was destroyed by adding amidosulfonic acid. The diazonium salt solution thus obtained was metered into a solution (pH 9-9.5) of 6.68 g (25.0 mmol) of 7-bromo-3-hydroxy-2-naphthoic acid and 1.48 g of 50% NaOH in 200 ml of water/acetonitrile (1:1), the pH of 8-9 being maintained by adding 7.45 g of 50% NaOH. The azo dye solution was stirred for a further hour.

Reduction: 1.34 g (16.8 mmol) of 50% NaOH were added (pH 8.9) to the solution of the azo dye, and heating was effected under nitrogen to 80° C. 8.71 g (50.0 mmol) of sodium dithionite were added in portions, the pH of the solution being kept above 8 by adding 2.77 g of 50% NaOH. After decolorization of the reaction solution, it was allowed to cool to room temperature and brought to pH 5.1 with concentrated HCl, a precipitate being obtained. The solid was filtered off with suction under nitrogen, washed with water and dried over NaOH in a desiccator.

Yield: 5.13 g of virtually colorless powder; m.p. 264-267° C. (decomposition)

¹H-NMR (D₆-DMSO): 7.50 (d; 1H, aromatic-H), 7.82 (s; 1H, aromatic-H), 8.00 (d; 1H, aromatic-H), 8.10 (s; 1H, aromatic-H), 9.1 (broad; 2H)

### Example 29

### 3,9,15,21-Tetrabromocyclo-2,9′:2′,9″:2″,9′″:2′″,9-quaternaphtho[1,2-d]oxazole

4.23 g (15.0 mmol) of 4-amino-7-bromo-3-hydroxy-2-naphthoic acid were introduced in portions into 100.9 g of 85% strength polyphosphoric acid at 135° C. with stirring and under nitrogen. The reaction solution was heated to 150° C. and stirred at this temperature for 24 hours. After the solution had been cooled to 120° C., it was added with stirring to 250 g of ice, a precipitate being obtained. The suspension was neutralized with 161 g of concentrated ammonia solution, cooling being effected by adding 127 g of ice. The precipitate was filtered off with suction and washed with water until the wash water had a neutral pH. The crude product was dried at 75° C. under reduced pressure (3.62 g). For purification, the brown crude product was dissolved in 127 g of 96-98% strength sulfuric acid. The solution was filtered over a glass frit, and 16.5 g of 50% strength sulfuric acid were slowly added, crystals forming. The solid was filtered off with suction over a glass frit, washed with 90% strength sulfuric acid and with water and dried at 75° C. under reduced pressure (1.25 g). In a second purification step, the solid was dissolved in 29.3 g of 100% strength sulfuric acid, and 0.59 g of 95-97% strength sulfuric acid and then 1.73 g of 50% strength sulfuric acid were slowly added. The crystals were filtered off with suction over a glass frit, washed with water and dried under reduced pressure at 80° C.

Yield: 0.50 g of yellow microcrystals

UV/Vis: λₘₐₓ=410, 332, 262, 228 nm in concentrated sulfuric acid

### Example 30

### Use as a Pigment Dispersant

### 3,4-Dicarboximidoperylene-9-carboxylic acid was prepared according to CAS 75:140549j (1971). Solsperse 12000 (from Avecia) was used as a suitable copper phthalocyanine-sulfonic acid. Solsperse 24000 was used as hyperdispersant.

a) 7.50 g of a transparent commercial perylene pigment, for example P.R.179 (L3885 BASF) were thoroughly dry-blended in each case with 225 mg of the synergistic agents 3,4-dicarboximidoperylene-9-carboxylic acid (sample 1), with 225 mg of Solsperse 12000 (sample 2) and with 225 mg of sulfonated cyclo-2,9′:2′,9″:2″,9′″:2′″,9-quaternaphtho[1,2-d]oxazole from Example 21 (sample 3). 7.725 g of a pigment without synergistic agent were used as a comparison (sample 4). The dispersing was effected in a 100 ml glass bottle using 27 ml of glass balls having a ball diameter of 3 mm. After addition of 1.2 g of the hyperdispersant Solsperse 24000 and 23.3 g of a high solids finish (acrylic resin with xylene and butyl acetate 1:1 as solvent, solids content 45%) to all samples, shaking was effected for 2 hours using a Skandex dispersing unit. The pastes were left to stand for 24 hours. Pastes 1a-3a had a thin consistency whereas comparative paste 4a had a thick consistency. The synergistic agent had a similar efficiency. After mixing of, in each case, 1.0 g of finish paste with 2.0 g of a white paste (comprising 20 g of TiO₂ dispersed in 60 g of unpigmented high solids finish, see above) and application of this paste by means of a knife coater to a metal sheet and subsequent baking (30 minutes, 130° C.), coating films were obtained. The coating films from 1a, 3a and 4a had a similar color whereas the film from paste 2a (containing blue phthalocyanine derivative) was substantially more opaque and bluer.
b) The abovementioned experiment was repeated using 10.0 g of a commercial pigment blue, for example P.B. 15:1 (L6930 BASF) instead of 7.5 g of P.R.179. The pastes 1b-3b had a low viscosity and a similarly good flowability. After production of the coating films, it is evident that the film from paste 1b (containing perylene derivative) appeared redder and more opaque, whereas the other films from pastes 2b, 3b, 4b gave an almost identical color impression when considered visually.

### Example 31

Organic layers were applied by vapor deposition, at a greatly reduced pressure (10⁻⁶ mbar) to a clean glass coated with indium tin oxide (ITO). A hole-conducting layer comprising N,N′-diphenyl-N,N′-bis(3-methylphenyl)-(1,1′-biphenyl)-4,4′-diamine (TPD), an emitter layer comprising cyclo-2,4′:2′,7″:2″,4′″:2′″,7-quaterbenzimidazole (CQBI), an electron-conducting layer comprising aluminum tri-(8-quinolinolate (Alq₃) and a cathode layer comprising aluminum were applied in succession by vapor deposition to the ITO layer (anode).

### Example 32

Organic layers were applied by vapor deposition, at a greatly reduced pressure (10⁻⁶ mbar) to a clean glass coated with indium tin oxide (ITO). A hole-conducting layer comprising

N,N′-di(1-naphthyl)-N,N′-diphenyl-4,4′-benzidine (α-NPB), an emitter layer consisting of the matrix material 4,4′-(biphenyl)-N,N′-dicarbazole (CBP) and the emitter platinum cyclo-2,4′:2′,7″:2″,4″′:2′″,7-quaterbenzimidazole (Pt-CQBI), a hole-blocking layer comprising 2,9-dimethyl-4,7-diphenylphenanthroline (BCP), an electron-conducting layer comprising aluminum tri-(8-quinolinolate) (Alq₃), an electron-injecting layer comprising lithium fluoride and a cathode layer comprising aluminum were applied in succession by vapor deposition to the ITO layer (anode), said emitter layer being applied by covaporization.

### Example 33

Organic layers were applied by vapor deposition, at a greatly reduced pressure (10⁻⁶ mbar) to a clean glass coated with indium tin oxide (ITO). A hole-injection layer comprising copper phthalocyanine (CuPc), a hole-conducting layer comprising N,N′-di(1-naphthyl)-N,N′-diphenyl-4,4′-benzidine (a-NPB), an emitter layer consisting of the matrix material 4,4′-(biphenyl)-N,N′-dicarbazole (CBP) and the emitter platinum cyclo-2,4′:2′,7″:2″,4′″:2′″,7-quaterbenzimidazole (Pt-CQBI), a hole-blocking layer comprising 2,9-dimethyl-4,7-diphenylphenanthroline (BCP), an electron-conducting layer comprising aluminum tri-(8-quinolinolate) (Alq₃), an electron-injecting layer comprising lithium fluoride and a cathode layer comprising aluminum were applied in succession by vapor deposition to the ITO layer (anode), said emitter layer being applied by covaporization.

### Example 34

Organic layers were applied by vapor deposition, under greatly reduced pressure (10⁻⁶ mbar) to a clean glass coated with indium tin oxide (ITO). A hole-injection layer comprising cyclo-2,9′:2′,9″:2″,9′″:2′″,9-quaternaphtho[1,2-d]oxazole, a hole-conducting layer comprising N,N′-di(1-naphthyl)-N,N′-diphenyl-4,4′-benzidine (α-NPB), an emitter layer consisting of the matrix material 4,4′-(biphenyl)-N,N′-dicarbazole (CBP) and the emitter platinum cyclo-2,4′:2′,7″:2″,4′″:2′″,7-quaterbenzimidazole (Pt-CQBI), a hole-blocking layer comprising 2,9-dimethyl-4,7-diphenylphenanthroline (BCP), an electron-conducting layer comprising aluminum tri(8-quinolinolate) (Alq₃), an electron-injecting layer comprising lithium fluoride and a cathode layer comprising aluminum were applied in succession by vapor deposition to the ITO layer (anode), said emitter layer being applied by covaporization.

### Example 35

Organic layers were applied by vapor deposition, under greatly reduced pressure (10⁻⁶ mbar) to a clean glass coated with indium tin oxide (ITO). A hole-injection layer comprising cyclo-2,9′:2′,9″:2″,9′″:2′″,9-quaternaphtho[1,2-d]oxazole, a hole-conducting layer comprising N,N′-di(1-naphthyl)-N,N′-diphenyl-4,4′-benzidine (α-NPB), an emitter layer consisting of the matrix material 4,4′-(biphenyl)-N,N′-dicarbazole (CBP) and the emitter iridium tris(2-phenylpyridine) (Ir(ppy)₃), a hole-blocking layer comprising 2,9-dimethyl-4,7-diphenylphenanthroline (BCP), an electron-conducting layer comprising aluminum tri(8-quinolinolate) (Alq₃), an electron-injecting layer comprising lithium fluoride and a cathode layer comprising aluminum were applied in succession by vapor deposition to the ITO layer (anode), said emitter layer being applied by covaporization.

### Example 36

Use example for compounds from examples 13 and 15 compared with the pigmentary UV absorber ZnO (Z-Cote; BASF)

Nanoparticulate ZnO (DE-A 19907704, EP-A 0449888) is available as pigmentary inorganic UV absorbers for cosmetics, finish and plastic. However, these have the disadvantage that, in the event of insufficient fineness, they scatter white light giving rise to a milky appearance. Insufficient fineness occurs when the particle size is too large, either the primary particles being too large or the dispersing state being insufficient. The claimed compounds have an absorption spectrum similar to that of ZnO but, the organic pigments scatter to a lesser extent in the formulations described.

### In LDPE

5 g of LDPE 1840 (from Basell) and 0.05 g of the samples from examples 1d, 2 and commercial zinc oxide (Z-Cote, from BASF) were plasticated in a Mini Extruder (MiniLab Micro Rheology Compounder, from Thermo Haake) at 170° C. and dispersed at this temperature for 5 minutes. The discharge was introduced into a mold having a temperature of 50° C. (2.5 cm diameter, 1.5 mm thickness) by means of a spray gun at 180° C. This injected sample was compressed to a thickness of 0.25 mm. Finally, the injected sample was pressed between two press plates at 180° C. by means of a steam press (from Wickert) to a thickness of 0.25 mm and was characterized visually and by UV spectroscopy. When observed visually, the impression molding, which contained the compounds from examples 1 and 2, had a higher transparency than the ZnO-containing sample, with otherwise similar absorbance.

### In an Automotive Clearcoat

60 mg of the compounds from examples 1 and 2 and zinc oxide (Z-Cote, BASF) were dispersed in a 10 ml glass bottle containing 6 g of SAZ balls (1-1.6 mm diameter) and 5.94 g of an automotive clear coat without UV absorber (Duraclear II, BASF Coatings, 50% solids content, butyl acetate solvent) for 60 minutes on a Skandex dispersing unit (from Lau) at level 2. A 150 μm film was applied by means of a manual spiral applicator and was baked for 30 minutes at 130° C. The films were assessed visually. With similar absorption, the compounds containing examples 1 and 2 showed less scattering of white light, which was evident from a less milky appearance.

### In a Cosmetic Formulation

8.00 g of dibutyl adipate (Cetiol b, from Cognis), 8.00 g of C₁₂₋₁₅ alkyl benzoate, 12.00 g of cocogylcerides (Myritol 331, from CA Erbslöh), 1.00 g of sodium cetearyl sulfate (Lanette E, from Cognis) and 4.00 g of lauryl glucoside, (Eumulgin VL75, from Cognis), 2.00 g of cetearyl alcohol (Lanette 0) and 1.00 g of Vitamin E Acetate (BASF) and 3.00 g of ethylhexyltriazone (Uvinul T150 BASF) were homogenized at 80° C.

Thereafter, the novel compounds from examples 1 and 2 and Z-Cote were added at this temperature and dispersed for 3 minutes using an Ultraturrax stirring rod. A dispersion which had a temperature of 80° C. and was obtained by homogenizing 3.00 g of glycerol, 0.05 g of EDTANa₂ (EDETA BD, from BASF), 0.20 g of allantoin, 0.30 g of xanthan gum (Keltrol, from Kleco), 1.50 g of magnesium aluminum silicate (Veegum Ultra, from Vanderbilt) and 50.45 g of distilled water at 80° C. was added to this dispersion. The combined dispersions were cooled to 40° C., and 0.50 g of citric acid, possibly fragrances and 1.00 g of a mixture of phenoxyethanol and alkylparabens (Phenonip, from Nipa) were added.

The formulation obtained can be used as a suncream.

The formulations, which contained the claimed compounds, had a similar absorption spectrum at the same film thickness but scatter less white light than the formulation containing ZnO (Z-Cote, BASF), which has esthetic advantages particularly in the case of dark-pigmented skin.

1-10. (canceled)

11. A light absorber, a material for hole injection layers in OLEDs, a light-emitting compound in OLED, a synergistic agent for dispersing pigments or for optical data storage composition comprising a composition comprising:
a cyclic compound represented by the formula (I) and at least one tautomeric structure thereof 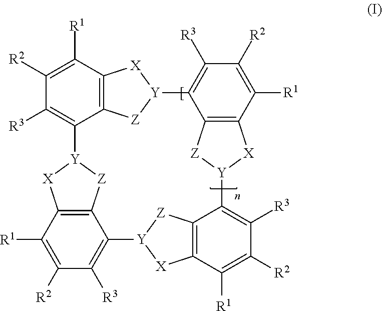
or at least one metal complex of the cyclic compound
or at least one complex of the cyclic compound with mineral acids, chloride, sulfate, bisulfate, phosphate, hydrogen phosphate, nitrate, BF₄⁻ or methanesulfonate being present as opposite ions X⁻ in the case of cationic cyclic structures,
wherein
n is a number in the range from 1 to 7,
X—Y—Z, in each case independently of one another, is O—C═N, N═C—O, NR⁵—C═N, N═C—NR⁵, N⁺R⁵₂—C═N, N═C—N⁺R⁵₂, O—C═N⁺R⁵, N⁺R⁵═C—O, S—C═N⁺R⁵, N⁺R⁵═C—S, S—C═N, or N═C—S,
R¹, R² and R³, in each case independently of one another, are H or a substituent selected from the group consisting of substituted or unsubstituted C₁₋₁₂-alkyl, substituted or unsubstituted C₁₋₁₂-alkanoyl, substituted or unsubstituted C₃₋₇-cycloalkyl, substituted or unsubstituted C₆₋₁₂-aryl, substituted or unsubstituted C₇₋₁₃-aralkyl, substituted or unsubstituted C₇₋₁₃-alkaryl, substituted or unsubstituted C₁₋₁₂-alkoxy, substituted or unsubstituted C₆₋₁₂-aryloxy, substituted or unsubstituted C₁₋₁₂-hydroxyalkyl, substituted or unsubstituted heterocycle, substituted or unsubstituted C₆₋₁₂-aroyl, hydroxyl, thiol, cyano, isocyano, nitro, ammonium, amino, phosphine, phosphine oxide, a derivative of silicon, C₂₋₁₂-alkynyl, C₂₋₁₂-alkenyl, wherein the double or triple bonds of the C₂₋₁₂-alkynyl and C₇₋₁₂-alkenyl are optionally linked directly to the cycloquater skeleton or are optionally in the chain, a carbamate of the formula —NH—CO—OR⁷, a substituted urea of the formula —NR⁷—CO—NR⁷₂, and an alkyl carbonate substituent of the formula —O—CO—OR⁷;
R¹ and R² and/or R² and R³, in each case independently of one another, also optionally form unsubstituted or substituted fused ring systems comprising from 1 to 3 rings, which optionally comprise hetero atom groups, or optionally form unsubstituted or substituted alkylene groups which are optionally interrupted by hetero atom groups, wherein the fused ring systems are optionally substituted;
wherein the oxygen atoms in R¹, R² and/or R³ are optionally replaced by sulfur atoms,
on average from 0.05 to 100% of R¹, R² and R³ present in the composition are not hydrogen,
or corresponding heterocyclic compounds wherein at least one group —CR¹═, —CR²═, and —CR³═ is replaced by —N═,
R⁵, in each case independently of one another, are H, unsubstituted or substituted C₁₋₁₂-alkyl, C₆₋₁₂-aryl, C₇₋₁₃-alkylaryl, unsubstituted or substituted C₁₋₁₂-alkanoyl, unsubstituted or substituted C₇₋₁₃-aryloyl, oligoethylene glycol having 1 to 6 oxygen atoms, oligoethylene glycol ether having 1 to 6 oxygen atoms, imidazoylmethyl or a corresponding radical wherein a nitrogen atom is substituted by a C₁₋₁₂-alkyl radical and optionally carry a positive charge and a C—H group in the ring are optionally replaced by C—(C₁₋₁₂-alkyl), or (1-C₄₋₆-lactam)methyl, which are optionally C₁₋₁₂-alkyl-substituted on the ring,
R⁷, in each case independently of one another, are H, C₁₋₁₂-alkyl or C₆₋₁₂-aryl.

12. The light absorber, a material for hole injection layers in OLEDs, a light-emitting compound in OLED, a synergistic agent for dispersing pigments or for optical data storage composition as claimed in claim 11, wherein the composition comprises the cyclic compound of the formula (I) or at least one metal complex of the cyclic compound,
wherein
n is 1,
X—Y—Z, in each case independently of one another, is O—C═N, N═C—O, NH—C═N, N═C—NH, S—C═N or N═C—S,
R¹, R² and R³, in each case independently of one another, are H or a substituent selected from the group consisting of substituted or unsubstituted C₁₋₁₂-alkyl, substituted or unsubstituted C₁₋₁₂-alkanoyl, substituted or unsubstituted C₃₋₇-cycloalkyl, substituted or unsubstituted C₆₋₁₂-aryl, substituted or unsubstituted C₇₋₁₃-aralkyl, substituted or unsubstituted C₇₋₁₃-alkaryl, substituted or unsubstituted C₁₋₁₂-alkoxy, substituted or unsubstituted C₆₋₁₂-aryloxy, substituted or unsubstituted C₁₋₁₂-hydroxyalkyl, substituted or unsubstituted heterocycle, substituted or unsubstituted C₆₋₁₂-aroyl, hydroxyl, thiol, cyano, isocyano, nitro, ammonium, amino, phosphine, phosphine oxide, and a derivative of silicon,
R¹ and R² and/or R² and R³, in each case independently of one another, also optionally form unsubstituted or substituted fused ring systems comprising from 1 to 3 rings, which optionally comprise hetero atom groups, or optionally form unsubstituted or substituted alkylene groups which are optionally interrupted by hetero atom groups,
on average from 1 to 12 of R¹, R² and R³ present in the cyclic compound are not hydrogen,
or corresponding heterocyclic compounds wherein at least one group —CR¹═, —CR²═ or —CR³═ is replaced by —N═.

13. The light absorber, a material for hole injection layers in OLEDs, a light-emitting compound in OLED, a synergistic agent for dispersing pigments or for optical data storage composition as claimed in claim 11, wherein the cyclic compound of the formula (I) is comprised in the composition in soluble, partly soluble or insoluble form in an application medium,
wherein the insoluble form optionally comprises solid solutions with other colorants.

14. The light absorber, a material for hole injection layers in OLEDs, a light-emitting compound in OLED, a synergistic agent for dispersing pigments or for optical data storage composition as claimed in claim 11, wherein all R¹ are the same, all R² are the same, and all R³ are the same.

15. The light absorber, a material for hole injection layers in OLEDs, a light-emitting compound in OLED, a synergistic agent for dispersing pigments or for optical data storage composition according to claim 11, wherein R¹, R² and R³ are identical.

16. The light absorber, a material for hole injection layers in OLEDs, a light-emitting compound in OLED, a synergistic agent for dispersing pigments or for optical data storage composition according to claim 11, wherein on average from 1 to 8 of R¹, R² and R³ present in the cyclic compound are not hydrogen.

17. A material for hole injection layers in OLEDs, a light-emitting compound in OLED, a synergistic agent for dispersing pigments or for optical data storage composition comprising a composition comprising:
a cyclic compound represented by the formula (I) and at least one tautomeric structure thereof 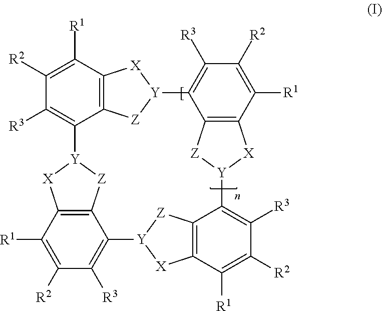
or at least one metal complex of the cyclic compound or at least one complex of the cyclic compound with mineral acids, chloride, sulfate, bisulfate, phosphate, hydrogen phosphate, nitrate, BF₄⁻ or methanesulfonate being present as opposite ions X⁻ in the case of cationic cyclic structures,
wherein
n is a number in the range from 1 to 7,
X—Y—Z, in each case independently of one another, is O—C═N, N═C—O, NR⁵—C═N, N═C—NR⁵, N⁺R⁵₂—C═N, N═C—N⁺R⁵₂, O—C═N⁺R⁵, N⁺R⁵═C—O, S—C═N⁺R⁵, N⁺R⁵═C—S, S—C═N, or N═C—S,
R¹, R² and R³, in each case independently of one another, are H or a substituent selected from the group consisting of substituted or unsubstituted C₁₋₁₂-alkyl, substituted or unsubstituted C₁₋₁₂-alkanoyl, substituted or unsubstituted C₃₋₇-cycloalkyl, substituted or unsubstituted C₆₋₁₂-aryl, substituted or unsubstituted C₇₋₁₃-aralkyl, substituted or unsubstituted C₇₋₁₃-alkaryl, substituted or unsubstituted C₁₋₁₂-alkoxy, substituted or unsubstituted C₆₋₁₂-aryloxy, substituted or unsubstituted C₁₋₁₂-hydroxyalkyl, substituted or unsubstituted heterocycle, substituted or unsubstituted C₆₋₁₂-aroyl, hydroxyl, thiol, cyano, isocyano, nitro, ammonium, amino, phosphine, phosphine oxide, a derivative of silicon, C₂₋₁₂-alkynyl, C₂₋₁₂-alkenyl, wherein the double or triple bonds of the C₂₋₁₂-alkynyl and C₂₋₁₂-alkenyl are optionally linked directly to the cycloquater skeleton or are optionally in the chain, a carbamate of the formula —NH—CO—OR⁷, a substituted urea of the formula —NR⁷—CO—NR⁷₂, and an alkyl carbonate substituent of the formula —O—CO—OR⁷;
R¹ and R² and/or R² and R³, in each case independently of one another, also optionally form unsubstituted or substituted fused ring systems comprising from 1 to 3 rings, which optionally comprise hetero atom groups, or optionally form unsubstituted or substituted alkylene groups which are optionally interrupted by hetero atom groups, wherein the fused ring systems are optionally substituted;
wherein the oxygen atoms in R¹, R² and/or R³ are optionally replaced by sulfur atoms,
on average from 0.05 to 100% of R¹, R² and R³ present in the composition are not hydrogen,
or corresponding heterocyclic compounds wherein at least one group —CR¹═, —CR²═, and —CR³═ is replaced by —N═,
R⁵, in each case independently of one another, are H, unsubstituted or substituted C₁₋₁₂-alkyl, C₆₋₁₂-aryl, C₇₋₁₃-alkylaryl, unsubstituted or substituted C₁₋₁₂-alkanoyl, unsubstituted or substituted C₇₋₁₃-aryloyl, oligoethylene glycol having 1 to 6 oxygen atoms, oligoethylene glycol ether having 1 to 6 oxygen atoms, imidazoylmethyl or a corresponding radical wherein a nitrogen atom is substituted by a C₁₋₁₂-alkyl radical and optionally carry a positive charge and a C—H group in the ring are optionally replaced by C—(C₁₋₁₂-alkyl), or (1-C₄₋₆-lactam)methyl, which are optionally C₁₋₁₂-alkyl-substituted on the ring,
R⁷, in each case independently of one another, are H, C₁₋₁₂-alkyl or C₆₋₁₂-aryl.

18. A material for hole injection layers in OLEDs, a light-emitting compound in OLED, a synergistic agent for dispersing pigments or for optical data storage composition comprising a composition comprising a cyclic compound of the formula (I) 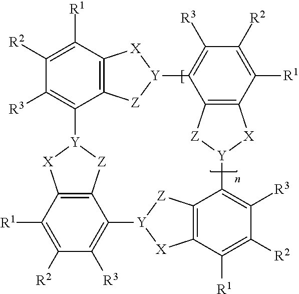
or at least one metal complex of the cyclic compound,
wherein
n is 1,
X—Y—Z, in each case independently of one another, is O—C═N, N═C—O, NH—C═N, N═C—NH, S—C═N or N═C—S,
R¹, R² and R³, in each case independently of one another, are H or a substituent selected from the group consisting of substituted or unsubstituted C₁₋₁₂-alkyl, substituted or unsubstituted C₁₋₁₂-alkanoyl, substituted or unsubstituted C₃₋₇-cycloalkyl, substituted or unsubstituted C₆₋₁₂-aryl, substituted or unsubstituted C₇₋₁₃-aralkyl, substituted or unsubstituted C₇₋₁₃-alkaryl, substituted or unsubstituted C₁₋₁₂-alkoxy, substituted or unsubstituted C₆₋₁₂-aryloxy, substituted or unsubstituted C₁₋₁₂-hydroxyalkyl, substituted or unsubstituted heterocycle, substituted or unsubstituted C₆₋₁₂-aroyl, hydroxyl, thiol, cyano, isocyano, nitro, ammonium, amino, phosphine, phosphine oxide, and a derivative of silicon,
R¹ and R² and/or R² and R³, in each case independently of one another, also optionally form unsubstituted or substituted fused ring systems comprising from 1 to 3 rings, which optionally comprise hetero atom groups, or optionally form unsubstituted or substituted alkylene groups which are optionally interrupted by hetero atom groups,
on average from 1 to 12 of R¹, R² and R³ present in the cyclic compound are not hydrogen,
or corresponding heterocyclic compounds wherein at least one group —CR¹═, —CR²═ or —CR³═ is replaced by —N═.
